(19) United States
(12) Patent Application Publication
(10) Pub. No.: US 2024/0216376 A1
(43) Pub. Date: Jul. 4, 2024

(54) COMBINATION THERAPIES FOR THE TREATMENT OF CANCER

(71) Applicant: Duke University, Durham, NC (US)

(72) Inventors: Kris Wood, Durham, NC (US); Kevin Lin, Durham, NC (US); Justine Rutter, Durham, NC (US)

(21) Appl. No.: 18/558,211

(22) PCT Filed: May 4, 2022
(86) PCT No.: PCT/US2022/027659

§ 371 Date: Oct. 31, 2023

Related U.S. Application Data
(60) Provisional application No. 63/183,806, filed on May 4, 2021.

(51) Int. Cl. A61K 31/517; A61K 31/497; A61K 31/506; A61P 35/02

(52) U.S. Cl. CPC A61K 31/517; A61K 31/497; A61K 31/506; A61P 35/02

(57) ABSTRACT

Described herein is a combination therapy for the treatment of a cancer in a subject. In one aspect, the therapy comprises selinexor and one or more second anti-cancer agents.

### CROSS-REFERENCE TO RELATED APPLICATIONS

This application claims priority to U.S. Provisional Patent Application No. 63/183,806 filed on May 4, 2021, which is incorporated by reference herein in its entirety.

### FEDERALLY SPONSORED RESEARCH

This invention was made with government support under grant numbers CA207083 and CA206348 awarded by the National Institutes of Health. The government has certain rights in the invention.

### TECHNICAL FIELD

Described herein is a combination therapy for the treatment of a cancer in a subject. In one aspect, the therapy comprises selinexor and one or more second anti-cancer agents.

### BACKGROUND

Selinexor is an orally bioavailable, selective inhibitor of nuclear export that recently received FDA approval for its use in the treatment of multiple myeloma and diffuse large B-cell lymphoma and is currently in early phase clinical trials for advanced solid and hematologic malignancies. There is particular interest in selinexor's potential for treating acute myeloid leukemia (AML), supported by a large body of preclinical evidence showing that inhibition of nuclear export promotes cell cycle arrest and apoptosis in AML cells. These therapeutic effects are evident, both in bulk leukemic populations and in leukemia-initiating cells (LICs) and can be enhanced by combining selinexor with existing chemotherapies. In patients with relapsed or refractory AML, selinexor was found to be tolerable and active, producing complete responses in a limited subset of patients. More recent trials have shown that, when administered in combination with other chemotherapies, selinexor is capable of inducing remission at a high rate in patients with relapsed or refractory AML, highlighting its emerging clinical activity in AML.

Mechanistically, selinexor blocks nuclear-cytoplasmic export by directly inhibiting the nuclear export protein chromosomal region maintenance 1 (CRM1—also known was exportin 1), which is responsible for facilitating RanGTP-dependent transport of nuclear export sequence (NES)-bearing cargos from the nucleus to the cytoplasm. Inhibition of CRM1 results in the nuclear accumulation of its substrates, among them: tumor suppressor proteins, cell cycle regulators, and DNA damage response proteins such as Rb, p53, p21, p27, FOXO3, BRCA1, CHK1, and RAD51. In the setting of cancer, these proteins, which require nuclear localization to serve their functions, are frequently dislocated to the cytoplasm due to the activation of oncogenic signaling or frank upregulation of CRM1; returning them to the nucleus restores their activity, thus providing a therapeutic, anti-cancer effect. However, CRM1 is not specific for protein clients with tumor suppressive activity. Unbiased proteomic studies have identified hundreds of CRM1 substrates in human cells. Accordingly, CRM1 inhibition may engage numerous cellular programs simultaneously, leaving open the possibility that selinexor treatment may also activate pro-oncogenic processes.

There are examples in the literature where treatment with an anti-cancer therapy elicits pleiotropic effects, including those that promote tumor survival. Treating neuroblastoma cells with the proteasome inhibitor bortezomib stabilizes the anti-apoptotic protein MCL-1, thereby diminishing sensitivity to taxol-induced apoptosis. Hypomethylating agents have been shown to activate oncogenic programs in Hodgkin's lymphoma, head and neck cancer, and lung cancer, consistent with longstanding observations that premalignant states can be transformed by global DNA hypomethylation events targeting oncogenic promoters. These drugs share an important feature with selinexor: while they may be specific to their respective targets, the targets themselves serve broad cellular processes that engage many aspects of the cancer cell, each of which may be impacted by drug treatment. Most studies fail to account for these secondary, on-target effects. As drugs targeting such processes-chromatin regulation, transcription, translation, and protein degradation, among others-become increasingly relevant, it will be important to recognize their tumor-promoting effects. In principle, if these drug-induced, pro-fitness pathways can be identified, their preemptive inhibition could enhance the effectiveness of drug. As a result, methods for treating, e.g., cancer may benefit from combination therapies that inhibit associated pro-fitness pathways.

### SUMMARY

One embodiment described herein is a method for treating a cancer in a subject in need thereof, the method comprising administering to the subject an effective amount of a first agent that inhibits cellular nucleus export and an effective amount of a second agent that inhibits Protein kinase B (Akt). In one aspect, the first agent inhibits exportin 1. In another aspect, the first agent comprises Selinexor, Eltanexor, or a combination thereof. In another aspect, the second agent inhibits Akt via inhibiting a G-protein coupled receptor (GPCR), a phosphoinositide 3-kinase (PI3K), or a combination thereof. In another aspect, the second agent inhibits Akt via inhibiting purinergic receptor P2RY2, phosphoinositide 3-kinase gamma (PI3K γ), or a combination thereof. In another aspect, the second agent comprises MK-2206 2HCl, Perifosine, GSK690693, AZD5363, Ipatasertib, Capivasertib, PF-04691502, AT7867, Tricirbine, CCT128930, A-674563, PHT-427, Miransertib, BAY1125976, Borussertib, Miransertib, Akti-1/2, Uprosertib, Afuresertib, AT13148, Oridonin, Miltefosine, Honokiol, TIC10 Analogue, Urolithin B, Resibufogenin, Cinobufagin, Daphnoretin, Loureirin A, Trigoneline, ML-9 HCl, ABTL-0812, Alobresib, Praeruptorin A, Oroxin B, SC66, Usnic acid, Scutellarin, Astragaloside IV, Deguelin, TIC10, Methyl-Hesperidin, IPI-549, TAS-117, ARQ-751, LY2780301, or a combination thereof. In another aspect, the second agent comprises Ipatasertib. In another aspect, the cancer comprises leukemia, lymphoma, myeloproliferative neoplasms, myelodysplastic syndromes, amyloidosis, Waldenstrom's macroglobulinemia, aplastic anemia, myeloma, or solid cancers. In another aspect, the cancer comprises acute myeloid leukemia (AML). In another aspect, the first agent is administered concurrently with the second agent to the subject. In another aspect, the second agent is administered to the subject after the first agent is administered. In another aspect, the second agent is administered at least 8 hours after the first agent is administered. In another aspect, the effective amount of the first agent comprises about 0.1 mg/kg to about 100 mg/kg, the effective amount of the second agent comprises about 0.1 mg/kg to about 100 mg/kg, or a combination thereof. In another aspect, the first agent and the second agent are administered as a single dosage form. In another aspect, the subject is a mammal. In another aspect, the second agent inhibits a pro-oncogenic effect of the first agent. In another aspect, the method decreases a number of CD45+ cancer cells in the subject compared to an administration of the first agent alone or the second agent alone. In another aspect, the method increases a time of survival of the subject compared to an administration of the first agent alone, the second agent alone, or cytarabine and doxorubicin. In another aspect, the method decreases a number of leukemia-initiating cells in the subject compared to an administration of cytarabine and doxorubicin.

Another embodiment described herein is a composition comprising: a first agent that inhibits cellular nucleus export; and a second agent that inhibits Akt; and one or more pharmaceutically acceptable excipients. In another aspect, the pharmaceutically acceptable excipients comprise buffering agents, solubilizers, solvents, antimicrobial preservatives, antioxidants, suspension agents, a tablet or capsule diluent, or a tablet disintegrant. In another aspect, the first agent inhibits exportin 1. In another aspect, the second agent inhibits Akt via inhibiting purinergic receptor P2RY2, phosphoinositide 3-kinase gamma (PI3K γ), or a combination thereof. In another aspect, the composition comprises about 1 mg to about 800 mg of the first agent, comprises about 1 mg to about 800 mg of the second agent, or a combination thereof. In another aspect, the second agent inhibits a pro-oncogenic effect of the first agent.

Another embodiment described herein is a kit comprising: a first agent that inhibits cellular nucleus export; a second agent that inhibits Akt; and one or more packages, receptacles, delivery devices, labels, or instructions for use.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-D show functional genomics and proteomics nominate AKT activation as a targetable consequence of selinexor treatment in accordance with one embodiment of the present disclosure. FIG. 1A. Experimental strategy for parallel assessment of cell-beneficial and cell-detrimental effects of nuclear export inhibition with the CRM1 inhibitor, selinexor. Pooled CRISPR-Cas9 screening in OCI-AML2 cells treated with selinexor reveals genetic modifiers of drug sensitivity. Reverse phase protein array (RPPA) analysis in selinexor treated OCI-AML2 cells reveals drug-responsive protein and phosho-protein expression. FIG. 1B. Selinexor depletion gene scores ranked from most deplete to most enriched in the selinexor versus vehicle treated populations. Scoring genes in the PI3K/AKT pathway are annotated as sensitizers (orange, depleted in the selinexor population) or resisters (blue, enriched in the selinexor population). Screens conducted as independent replicates with n=5 sgRNAs per gene. FIG. 1C. Volcano plot depicting differential expression for 160 RPPA probes following 48 hours of selinexor treatment relative to statistical significance in dataset. Annotated probes comprise pathways with decreased expression (blue) or increased expression (orange). RPPA expression analysis conducted as n=3 independent experiments. FIG. 1D. Schematic relating PI3K/AKT pathway members to selinexor depletion gene scores and RPPA expression. Genes scoring as selinexor sensitizers are shaded in orange; genes scoring as selinexor resisters are shaded in blue; genes included in the library but inert to selinexor sensitivity are shaded in gray; genes absent from library not shaded. Phosphorylated proteins with selinexor-induced increased (orange) or decreased (blue) RPPA expression are indicated.

FIG. 2A-E show Selinexor treatment activates PI3K/AKT signaling in AML cells in accordance with one embodiment of the present disclosure. FIG. 2A. Immunoblot depicting protein levels of phosphorylated and total PI3K/AKT pathway members following treatment of a panel of AML cell lines with selinexor. Selinexor was dosed at the following concentrations for each cell line: OCI-AML2 (200 nM), MOLM-13 (75 nM), MV; 411 (50 nM), HL-60 (300 nM), OCI-AML3 (250 nM). FIG. 2B. Immunoblot depicting protein levels of phosphorylated AKT at T308 and S473 in OCI-AML2 cells treated with Selinexor for indicated duration. FIG. 2C. Immunoblot depicting protein levels of phosphorylated AKT at T308 and S473 in dsRed+ MLL-AF9 cells from mice treated with Selinexor for indicated duration. FIG. 2D. Immunoblot depicting protein levels of phosphorylated AKT at T308 and S473 in OCI-AML2 cells treated with a panel of pipeline or standard-of-care AML chemotherapies for 24 hours. FIG. 2E. Immunoblot depicting protein levels of CRM1 and phosphorylated AKT at T308 in OCI-AML2 cells with doxycycline (dox) inducible shRNAs targeting XPO1 versus scrambled shRNA control. Cells were exposed to dox (75 ng/mL) for indicated durations prior to collection.

FIG. 3A-E show FACS-based CRISPR/Cas9 screening identifies genetic determinants of selinexor-induced AKT activation in accordance with one embodiment of the present disclosure. FIG. 3A. FACS-based CRISPR/Cas9 screening strategy to identify genetic modifiers of AKT phosphorylation in selinexor-treated AML cells. sgRNA library transduced OCI-AML2 cells were treated with selinexor for 48 hours, fixed/permeabilized and stained with phosphorylated AKT T308 primary antibody followed by Alexa Flour 488 conjugated secondary antibody. Stained cells were then sorted according to phosphorylated AKT T308 expression into high-expressing cells (top sort) and low-expressing cells (bottom sort). Genomic DNA was extracted and sgRNA barcodes were amplified and indexed prior to deep sequencing. The FACS screen gene score (FSGS) enumerates genes whose sgRNA representatives were enriched in the top or bottom sorted populations. FIG. 3B. Histogram depicting distribution of phosphorylated AKT T308 expression in parental OCI-AML2 cells treated with vehicle or selinexor for 48 hours. FIG. 3C. Histogram depicting distribution of phosphorylated AKT T308 expression in sgRNA library transduced OCI-AML2 cells treated with vehicle or selinexor for 48 hours. Gates defining top (blue) and bottom (orange) sorted population in selinexor treated cells are indicated. FIG. 3D. Scatterplot depicting replicate FSGS values. Scoring genes enriched in the bottom sort (orange) or the top sort (blue) are annotated. LacZ. EGFP and luciferase targeting controls indicated in white. Screens conducted as independent replicates with n=4 sgRNAs per gene. FIG. 3E. Gene ontology (GO) analysis of scoring genes enriched in the bottom sort with a p-value<5×10⁻⁴. GO performed using Enrichr.

FIG. 4A-H show Selinexor-induced upregulation of P2RY2 drives activation of AKT in accordance with one embodiment of the present disclosure. FIG. 4A. Scatterplot depicting replicate-averaged FSGS compared to differential gene expression (DE) score with selinexor treatment. RNA-seq analysis of selinexor versus vehicle treated OCI-AML2 and MOLM-13 cells yielded DE for each cell line. Plotted DE score is the average DE across the two cell lines. Blue shaded region denotes genes that were both transcriptionally upregulated by selinexor treatment and yielded a high FSGS score in the FACS-based CRISPR/Cas9 screen. RNA-seq conducted as n=3. FIG. 4B. Relative expression of P2RY2 across a panel of selinexor-treated AML cell lines compared to DMSO control. FIG. 4C. Immunoblot depicting protein levels of phosphorylated AKT at T308 and S473, cleaved-parp and immunoprecipitated GTP-bound Ras in OCI-AML2 cells with dox-inducible shRNAs targeting P2RY2 versus scrambled shRNA control. Cells were exposed to dox (75 ng/ml) for 48 hours and treated with either vehicle or selinexor for 36 hours. FIG. 4D. Immunoblot depicting protein levels of phosphorylated AKT at T308 and S473 in OCI-AML2 cells following treatment of pertussis toxin (Ptx 100 ng/ml), AR-C 118925XX (AR-C 2.5 UM) or selinexor alone and in combination for 36 hours. FIG. 4E. Immunoblot depicting protein levels of phosphorylated AKT at T308 and S473 in OCI-AML2 cells with dox-inducible shRNAs against PIK3CG and PIK3R5 versus scrambled shRNA control. Cells were exposed to dox (75 ng/ml) for 48 hours and treated with either vehicle or 200 nM selinexor for 36 hours. FIG. 4F. Immunoblot depicting active Ras following co-immunoprecipitation of Ras-GTP with GST-Raf1-Ras-binding domain (RBD) fusion proteins in a panel of selinexor-treated AML cell lines. Total Ras in input lysate shown as control. FIG. 4G. Immunoblot depicting active Ras as in FIG. 4F in OCI-AML2 cells treated with AR-C 118925XX (2.5 UM) and Selinexor (200 nM), alone and in combination. FIG. 4H Immunoblot depicting protein levels of phosphorylated AKT at T308 and S473 in OCI-AML2 cells co-expressing dox-inducible shRNAs against NRAS and KRAS versus scrambled shRNA control. Cells were exposed to dox (75 ng/ml) for 48 hours and treated with either vehicle or selinexor for 36 hours.

FIG. 5A-G show inhibition of AKT sensitizes AML cells to selinexor treatment in accordance with one embodiment of the present disclosure. FIG. 5A. Relative GI50 values of selinexor in combination with AKT inhibitors across a panel of AML cell lines. Relative selinexor GI50 value defined as (GI50 selinexor+AKT inhibitor)/(GI50 selinexor alone). Background AKT inhibitors dosed by cell line (OCI-AML2, 5 UM; MOLM-13, 3 UM; MV4; 11, 3 μM; HL-60, 5 UM; OCI-AML3, 3 μM; Kasumi-1, 3 μM; U937, 3 μM; THP-1, 5M). FIG. 5B. Time-to-progression assay of OCI-AML2 cells treated with 200 nM selinexor, 5 μM MK-2206 or the two drugs in combination. FIG. 5C. Relative GI50 values of selinexor in combination with PI3K-α/β/δ/γ specific inhibitors across a panel of AML cell lines. Relative selinexor GI50 value defined as (GI50 selinexor+PI3K inhibitor)/(GI50 selinexor alone). Background PI3K inhibitors dosed by cell line (OCI-AML2, 1 μM; HL-60, 4 μM; MOLM-13, 2 μM; MV4; 11, 2 μM; THP-1, 1 μM). FIG. 5D Immunoblot depicting protein levels of cleaved-caspase 3 and cleaved-parp across a panel of AML cell lines treated with selinexor (OCI-AML2, 200 nM; MOLM-13, 75 nM; MV4; 11, 50 nM; OCI-AML3, 250 nM; HL-60, 300 nM), MK-2206 (OCI-AML2, 5 μM; MOLM-13, 3 μM; MV4; 11, 3 μM; OCI-AML3, 3 μM; HL-60, 5 μM) or the combination. FIG. 5E. Relative selinexor GI50 values in OCI-AML2 and MOLM-13 cells harboring shRNAs against BAX or GFP control and treated with selinexor alone (OCI-AML2, 200 nM; MOLM-13 75 nM) or in combination with MK-2206. Relative selinexor GI50 value defined as (GI50 selinexor+MK-2206)/(GI50 selinexor alone). FIG. 5F. Bliss synergy scores for 32 primary patient samples treated with a drug-dilution matrix. Negative values denote absolute antagonism. Positive values denote synergy. Notable synergy demarked by line of Bliss=1. FIG. 5G. Methylcellulose colony formation assays performed using primary AML patient samples and treated with selinexor (#11: 5 nM, #17: 5 nM) and/or ipatasertib (#11: 0.75 UM, #17: 5 μM) as indicated. Colonies were stained and counted at 14 days.

FIG. 6A-K show combined inhibition of CRM1 and AKT prolongs survival in murine models of AML in accordance with one embodiment of the present disclosure. FIG. 6A. Kaplan-Meier survival curves of OCI-AML2 cell line xenograft NSG mice treated with vehicle, ipatasertib, selinexor or the two drugs in combination. Study conducted with n=10 mice per cohort. FIG. 6B. Kaplan-Meier survival curves of AML patient-derived xenograft (PDX1) NOG-EXL mice treated with vehicle, ipatasertib, selinexor or the drug combination. Duration treatment for all conditions depicted as blue bar. Study conducted with n=6 mice per cohort. Statistical significance determined by log-rank (Mantel-Cox) test. FIG. 6C. FACS quantification of human CD45+ leukemic blast cells from murine bone marrow aspirates of PDX1 engrafted NOG-EXL mice. FIG. 6D. Kaplan-Meier survival curves of MLL-AF9 syngeneic murine model of AML treated with vehicle, ipatasertib, selinexor or the drug combination. Duration of treatment for all conditions denoted with blue boxes along x-axis. Study conducted with n=5 mice per cohort. Statistical significance determined by log-rank (Mantel-Cox) test. FIG. 6E. FACS quantification of MLL-AF9 dsRed+ leukemic blast cells from murine bone marrow aspirates following treatment with conditions indicated in FIG. 6D. FIG. 6F. Kaplan-Meier survival curves of MLL-AF9 syngeneic murine model of AML treated with vehicle, standard-of-care chemotherapy, or the combination of ipatasertib plus selinexor. Duration of treatment for all conditions denoted with blue boxes along x-axis. Study conducted with n=8 mice per cohort. Statistical significance determined by log-rank (Mantel-Cox) test. FIG. 6G. FACS quantification of MLL-AF9 dsRed+ leukemic blast cells from murine bone marrow aspirates following treatment with conditions indicated in FIG. 6F. FIG. 6H FACS quantification of MLL-AF9 dsRed+ leukemic blast cells in spleen upon disease relapse following standard-of-care chemotherapy versus the ipatasertib plus selinexor drug combination. FIG. 6I. FACS quantification of MLL-AF9 dsRed+ leukemic blast cells in bone marrow upon disease relapse following standard-of-care chemotherapy versus the ipatasertib plus selinexor drug combination. FIG. 6J. Limiting dilution assay performed on MLL-AF9 cells isolated from primary mice treated with either standard chemotherapy (cytarabine, 100 mg/kg and doxorubicin, 1 mg/kg) or the combination of Selinexor (15 mg/kg) and Ipatasertib (65 mg/kg) for 24 hrs and reinjected into secondary recipient mice (n=5 mice per cohort). FIG. 6K. Determination of leukemia-initiating cell (LIC) frequency with a 95% confidence interval (CI) in each group using extreme limiting dilution analysis (ELDA). Statistical significance determined by a chi-squared (χ²) test.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T. U) and standard single letter amino acids (A, C, D, E, F, G, H, I, K, L, M, N, P, Q, R, S, T, V, W, or Y) are used herein.

As used herein, the terms such as “include,” “including.” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “formulation” and “composition” can be used interchangeably and refer to a combination of at least two ingredients. In some embodiments, at least one ingredient may be an active agent or otherwise have properties that exert physiologic activity when administered to a subject. As used herein, “therapeutic composition” and “pharmaceutical composition” can be used interchangeably and refer to a combination of at least two ingredients.

Described herein are studies using unbiased functional genomics and proteomics that identified activation of a P2RY2-PI3Kγ-AKT signaling pathway as a consequence of selinexor treatment in cancer cells, such as AML cells. The inventors found that inhibition of this pathway strongly potentiates the effect of selinexor, both in vitro, using cell lines and patient-derived primary cultures, and in vivo, using cell line and patient-derived xenografts together with genetically-engineered murine models of AML. More generally, these findings suggest that common conceptions of how anticancer therapies affect cancer cells are incomplete, and that a more holistic understanding of how these treatments unexpectedly promote cancer cell fitness could uncover hidden therapeutic opportunities.

Accordingly, one aspect of the present disclosure provides a method of treating and/or preventing a cancer in subject, the method comprising, consisting of, or consisting essentially of administering to the subject a therapeutically effective amount of a first agent that is a selective inhibitor of nuclear export and a therapeutically effective amount of a second agent that modulates Akt (also known as Protein Kinase B).

In one embodiment, the first agent comprises Selinexor.

In another embodiment, the second agent is selected from the group consisting of MK-2206 2HCl, Perifosine, GSK690693, Ipatasertib, Capivasertib, PF-04691502, AT7867, Tricirbine, CCT128930, A-674563, PHT-427, Miransertib, BAY1125976, Borussertib, Miransertib, Akti-1/2, Uprosertib, Afuresertib, AT13148, Oridonin, Miltefosine, Honokiol, TIC10 Analogue, Urolithin B, Resibufogenin, Cinobufagin, Daphnoretin, Loureirin A, Trigoneline, ML-9 HCl, ABTL-0812, Alobresib, Praeruptorin A, Oroxin B. SC66, Usnic acid, Scutellarin, Astragaloside IV, Deguelin, TIC10, Methyl-Hesperidin, and the like. In one embodiment, the second agent comprises Ipatasertib.

In some embodiments, the first agent is administered prior to the second agent. In another embodiment, the first agent is administered concurrently with the second agent. In other embodiments, the first agent is administered after the second agent.

In other embodiments, the cancer comprises a leukemia. In one embodiment, the cancer comprises acute myeloid leukemia (AML).

### 1. Methods for Treating Cancers

Disclosed herein are methods for treating a cancer. The method can include administering to the subject an effective amount of a first agent that inhibits cellular nucleus export and an effective amount of a second agent that inhibits Protein kinase B (Akt).

The disclosed methods can be used to treat a number of different cancers. For example, the method can be used to treat cancers that are suitable for treatment with the first agent. In other words, the methods can be used to treat cancers where inhibition of cellular nucleus export is advantageous for treatment. Example cancers include, but are not limited to, leukemia, lymphoma, myeloproliferative neoplasms, myelodysplastic syndromes, amyloidosis, Waldenstrom's macroglobulinemia, aplastic anemia, myeloma, and solid cancers. In some embodiments, the cancer is a hematological malignancy. In some embodiments, the cancer includes leukemia, lymphoma, myeloproliferative neoplasms, myelodysplastic syndromes, amyloidosis, Waldenstrom's macroglobulinemia, aplastic anemia, or myeloma. In some embodiments, the cancer includes leukemia or lymphoma. In some embodiments, the cancer includes leukemia. In some embodiments, the cancer includes acute myeloid leukemia (AML).

The subject that can be administered the first agent and the second agent can be any type of animal. Typically, the subject is a mammal. Accordingly, in some embodiments, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In some embodiments, the subject is a primate. In some embodiments, the subject is a human.

The disclosed methods can achieve advantageous cancer treatment in subjects. For example, the method can decrease a number of cancer cells, such as CD45+ cancer cells, in the subject compared to an administration of the first agent alone or the second agent alone. The method can decrease the number of cancer cells in the subject at least 2-fold, at least 3-fold, at least 5-fold, at least 10-fold, at least 15-fold, at least 20-fold, or at least 30-fold compared to an administration of the first agent alone or the second agent alone. In some embodiments, the method decreases the number of CD45+ leukemic cells as described above.

Furthermore, the method can increase the time of survival of the subject compared to an administration of the first agent alone, the second agent alone, or an administration of a standard-of-care chemotherapy. An example standard-of-care chemotherapy includes, but is not limited to, a combination of cytarabine and doxorubicin. The method can increase the survival of the subject by at least 5 days, at least 10 days, at least 15 days, at least 30 days, at least 2 months, at least 4 months, at least 6 months, or at least 1 year compared to an administration of the first agent alone, the second agent alone, or an administration of a standard-of-care chemotherapy. In some embodiments, the method provides cancer remission in the subject for at least 100 days following administration.

The method can also decrease a number of leukemia-initiating cells in the subject compared to an administration of a standard-of-care chemotherapy, such as cytarabine and doxorubicin. For example, the method can decrease the number of leukemia-initiating cells in the subject by at least 5-fold, at least 10-fold, at least 20-fold, at least 35-fold, at least, 30-fold, or at least 35-fold compared to an administration of a standard-of-care chemotherapy. In some embodiments, the method decreases the number of leukemia-initiating cells in the subject by about 5-fold to about 35-fold, such as about 10-fold to about 35-fold or about 20-fold to about 30-fold compared to an administration of a standard-of-care chemotherapy.

### A. First Agent

The first agent inhibits cellular nucleus export. For example, the first agent can inhibit certain proteins that regulate export of molecules, such as proteins and nucleic acids, out of a cell's nucleus to the cytoplasm. Inhibiting can include, e.g., binding to a nuclear export protein and altering the nuclear export protein's native function. An example protein that regulates nuclear export includes exportin-1. Accordingly, in some embodiments, the first agent inhibits exportin-1.

The first agent may be any suitable molecule or compound that can inhibit cellular nucleus export. Examples include, but are not limited to, small molecule drugs and RNA, such as siRNA and shRNA. The first agent may be a small molecule drug. In some embodiments, the first agent includes Selinexor, Eltanexor, or a combination thereof. In some embodiments the first agent includes Selinexor or Eltanexor. In some embodiments, the first agent includes Selinexor.

### B. Second Agent

The second agent inhibits Akt. Akt can include Akt kinases 1, 2, 3, and a combination thereof. The second agent can inhibit Akt directly, e.g., binding to Akt, or can inhibit Akt indirectly, such as by inhibiting other molecules that can lead to Akt signaling. For example, it has been found that administration of the first agent can activate phosphoinositide 3-kinase gamma (PI3K γ)-dependent Akt signaling through the upregulation of purinergic receptor P2RY2. This signaling activated by the first agent can lead to pro-oncogenic effects in the subject. Accordingly, the second agent can inhibit a pro-oncogenic effect of the first agent, e.g., by inhibiting Akt. In addition, in some embodiments, the second agent inhibits Akt via inhibiting purinergic receptor P2RY2, phosphoinositide 3-kinase gamma (PI3K γ), or a combination thereof. The second agent can also inhibit other proteins that result in Akt signaling. For example, the second agent can inhibit Akt via inhibiting g-protein coupled receptors (GPCRs) and/or phosphoinositide 3-kinases (PI3Ks) that can be upstream activators of Akt.

The second agent may be any suitable molecule or compound that can inhibit Akt. Examples include, but are not limited to, small molecule drugs and RNA, such as siRNA and shRNA. The second agent may be a small molecule drug. In some embodiments, the second agent includes MK-2206 (e.g., MK-2206 2HCl), Perifosine, GSK690693, AZD5363, Ipatasertib, Capivasertib, PF-04691502, AT7867, Tricirbine, CCT128930, A-674563, PHT-427, Miransertib, BAY1125976, Borussertib, Miransertib, Akti-1/2, Uprosertib, Afuresertib, AT13148, Oridonin, Miltefosine, Honokiol, TIC10 Analogue, Urolithin B, Resibufogenin, Cinobufagin, Daphnoretin, Loureirin A, Trigoneline, ML-9 HCl, ABTL-0812, Alobresib, Praeruptorin A, Oroxin B, SC66, Usnic acid, Scutellarin, Astragaloside IV, Deguelin, TIC10, Methyl-Hesperidin, IPI-549, TAS-117, ARQ-751, LY2780301, or a combination thereof. In some embodiments, the second agent includes MK-2206, GSK690693, AZD5363, TAS-117, ARQ-751, LY2780301, Ipatasertib, or a combination thereof. In some embodiments, the second agent includes MK-2206, GSK690693, AZD5363, or Ipatasertib. In some embodiments, the second agent includes Ipatasertib.

### C. Administration

The methods may include a “therapeutically effective amount” or a “prophylactically effective amount” of the first agent and the second agent (and compositions thereof). A “therapeutically effective amount” refers to an amount effective, at dosages and for periods of time necessary, to achieve the desired therapeutic result. A therapeutically effective amount of the first agent and the second agent can be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount can be less than the therapeutically effective amount.

For example, a therapeutically effective amount may be in the range of 1 mg to about 1000 mg of one or more of the agents (or compositions thereof) described herein. In one aspect, the therapeutically effective amount is about 5 mg to about 400 mg, including all integers and fractions within the range. In another aspect, the therapeutically effective amount is about: 2.5 mg, 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg of one or more of the agents described herein.

The first agent and the second agent can be administered at varying dosages to achieve an effective amount. For example, the effective amount of the first agent can include about 0.1 mg/kg to about 100 mg/kg, such as about 0.5 mg/kg to about 80 mg/kg, about 1 mg/kg to about 50 mg/kg, about 2 mg/kg to about 30 mg/kg, about 3 mg/kg to about 25 mg/kg, about 1 mg/kg to about 20 mg/kg, or about 1 mg/kg to about 15 mg/kg. In addition, the effective amount of the second agent can include about 0.1 mg/kg to about 100 mg/kg, such as about 0.5 mg/kg to about 80 mg/kg, about 1 mg/kg to about 50 mg/kg, about 2 mg/kg to about 30 mg/kg, about 3 mg/kg to about 25 mg/kg, about 1 mg/kg to about 20 mg/kg, or about 1 mg/kg to about 15 mg/kg. In some embodiments, the effective amount of the first agent comprises about 0.1 mg/kg to about 100 mg/kg, the effective amount of the second agent comprises about 0.1 mg/kg to about 100 mg/kg, or a combination thereof.

The first agent and the second agent (and compositions thereof) can be administered to the subject in different sequences and/or timing. For example, the first agent and the second agent can be administered to the subject concurrently. However, in some embodiments, the second agent can be administered to the subject after the first agent has been administered. For example, the second agent can be administered at least 30 minutes, 1 hour, 2 hours, 4 hours, 8 hours, 10 hours, 12 hours, 18 hours, or 24 hours after the first agent is administered. In some embodiments, the second agent is administered at least 6 hours after the first agent is administered. In some embodiments, the second agent is administered at least 7 hours after the first agent is administered. In some embodiments, the second agent is administered at least 8 hours after the first agent is administered. In some embodiments, the second agent is administered about 30 minutes to about 24 hours after the first agent is administered, such as about 1 hour to about 12 hours, about 2 hours to about 10 hours, about 6 hours to about 12 hours, or about 8 hours to about 18 hours after the first agent is administered.

In still other embodiments, the second agent can be administered to the subject prior to the first agent being administered. For example, the second agent can be administered to the subject about 5 hours, about 2 hours, about 1 hour, about 30 minutes, about 15 minutes, or about 5 minutes prior to the first agent being administered. Accordingly, in some embodiments, the second agent is administered to the subject about 5 minutes to about 5 hours prior to the first agent being administered.

The first agent and the second agent can be administered, for example, 1×, 2×, 3×, 4×, 5×, 6×, or even more times per day. The first agent and the second agent can be administered, for example, for 1, 2, 3, 4, 5, 6, 7 days, or even longer. One or more dosage forms (that include the first agent and the second agent) can be administered, for example, for 1, 2, 3, 4 weeks, or even longer. One or more dosage forms can be administered, for example, for 1, 2, 3, 4, 5, 6, 7, 8, 9, 10, 11, 12 months, 1 year, 2, years, 3 years, 4 years, 5 years, over 5 years, a decade, multiple decades, or even longer. One or more dosage forms can be administered at a regular interval until the subject or subject in need thereof, does not require treatment, prophylaxis, or amelioration of any disease or condition.

In one embodiment, the first agent and the second agent (and compositions thereof) can be administered as dosage forms in various regimens, including one dose per day (QD), two doses per day (BID), three doses per day (TID), or four times per day (QID) to achieve a total daily dosage. In another embodiment, any of the foregoing doses comprise a total daily dosage.

The first agent and the second agent can be administered via a variety of routes. Typical delivery routes include parenteral administration, e.g., intradermal, intramuscular, or subcutaneous delivery. Other routes include oral administration, intranasal, intravaginal, transdermal, intravenous, intraarterial, intratumoral, intraperitoneal, and epidermal routes. In some embodiments, the first agent, the second agent, or both are administered subcutaneously, intradermally, intramuscularly, or intravenously. In some embodiments, the first agent, the second agent, or both are administered orally.

### 2. Compositions

Also disclosed herein are compositions that can, e.g., be used for treating a cancer. The composition can include a first agent that is an inhibitor of cellular nucleus export; a second agent that inhibits Akt; and one or more pharmaceutically acceptable excipients. The description of the first agent and the second agent above may be applied to the disclosed compositions.

In some embodiments, the composition is a single dosage form. The dosage form can include the first agent and the second agent homogeneously throughout, or the dosage form can include the first agent and the second agent in specific, distinct regions of the dosage form. For example, the dosage form may include the first agent and the second agent in distinct regions such that they are released from the dosage form at different rates following administration. In some embodiments, the dosage form is a controlled release formulation.

In some embodiments, the pharmaceutically acceptable excipients include buffering agents, solubilizers, solvents, antimicrobial preservatives, antioxidants, suspension agents, a tablet or capsule diluent, a tablet disintegrant, or a combination thereof. In some embodiments, the pharmaceutically acceptable excipients include buffering agents, solubilizers, solvents, antimicrobial preservatives, antioxidants, suspension agents, a tablet or capsule diluent, or a tablet disintegrant.

The composition can include the first agent and the second agent at varying amounts. For example, the compositions can include about 1 mg to about 800 mg of the first agent, such as about 1 mg to about 600 mg, about 1 mg to about 500 mg, about 10 mg to about 500 mg, about 10 mg to about 400 mg, or about 1 mg to about 300 mg. In addition, the composition can include about 1 mg to about 800 mg of the second agent, such as about 1 mg to about 600 mg, about 1 mg to about 500 mg, about 10 mg to about 500 mg, about 10 mg to about 400 mg, or about 1 mg to about 300 mg. In some embodiments, the composition comprises about 1 mg to about 800 mg of the first agent, comprises about 1 mg to about 800 mg of the second agent, or a combination thereof.

### 3. Pharmaceutical Compositions

The disclosed agents and compositions thereof can be incorporated into pharmaceutical compositions suitable for administration to a subject (such as a patient, which may be a human or non-human). The description of the first agent, the second agent, and compositions thereof above may be applied to the disclosed pharmaceutical compositions. The pharmaceutical composition can be prepared for administration to a subject. Such pharmaceutical compositions can be administered in dosages and by techniques well known to those skilled in the medical, veterinary, and pharmaceutical arts taking into consideration such factors as the age, sex, weight, and condition of the particular subject, and the route of administration.

The pharmaceutical compositions and formulations can include pharmaceutically acceptable carriers. The term “pharmaceutically acceptable carrier.” as used herein, means a non-toxic, inert solid, semi-solid or liquid filler, diluent, encapsulating material, or formulation auxiliary of any type. Exemplary materials that can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the agents and their pharmaceutically acceptable salts can be formulated for administration by, for example, injection, inhalation (either through the mouth or the nose), solid dosing, eye drop, in a topical oil-based formulation, implants, oral, buccal, parenteral, or rectal administration. Techniques and formulations generally may be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered, and the form of the composition, will dictate the type of carrier to be used. The composition can be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50% to about 90%.

Suitable lubricants include, but are not limited to, silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition typically is about 5% to about 10%.

Suitable binders include, but are not limited to, polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition typically is about 5% to about 50%.

Suitable disintegrants include, but are not limited to, agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition typically is about 0.1% to about 10%.

Suitable colorants include, but are not limited to, a colorant such as an FD&C dye. When used, the amount of colorant in a systemic or topical composition typically is about 0.005% to about 0.1%.

Suitable flavors include, but are not limited to, menthol, peppermint, and fruit flavors. The amount of flavor(s), when used, in a systemic or topical composition typically is about 0.1% to about 1.0%.

Suitable sweeteners include, but are not limited to, aspartame and saccharin. The amount of sweetener(s) in a systemic or topical composition typically is about 0.001% to about 1%.

Suitable antioxidants include, but are not limited to, butylated hydroxyanisole (“BHA”), butylated hydroxytoluene (“BHT”), and vitamin E. The amount of antioxidant(s) in a systemic or topical composition typically is about 0.1% to about 5%.

Suitable preservatives include, but are not limited to, benzalkonium chloride, methyl paraben and sodium benzoate. The amount of preservative(s) in a systemic or topical composition typically is about 0.01% to about 5%.

Suitable glidants include, but are not limited to, silicon dioxide. The amount of glidant(s) in a systemic or topical composition typically is about 1% to about 5%.

Suitable solvents include, but are not limited to, water, isotonic saline, ethyl oleate, glycerin, hydroxylated castor oils, alcohols such as ethanol, and phosphate buffer solutions. The amount of solvent(s) in a systemic or topical composition typically is from about 0% to about 100%.

Suitable suspending agents include, but are not limited to, AVICEL RC-591 (from FMC Corporation of Philadelphia, PA) and sodium alginate. The amount of suspending agent(s) in a systemic or topical composition typically is about 1% to about 8%.

Suitable surfactants include, but are not limited to, lecithin, Polysorbate 80, and sodium lauryl sulfate, and the TWEEN® detergents. Suitable surfactants include, but are not limited to, those disclosed in the C.T.F.A. Cosmetic Ingredient Handbook, 1992, pp. 587-592; Remington's Pharmaceutical Sciences, 15th Ed. 1975, pp. 335-337; and Mccutcheon's Volume 1, Emulsifiers & Detergents, 1994, North American Edition, pp. 236-239. The amount of surfactant(s) in the systemic or topical composition typically is about 0.1% to about 5%.

Although the amounts of components in the systemic compositions may vary depending on the type of systemic composition prepared, in general, systemic compositions include about 0.01% to about 50% of an active compound and about 50% to about 99.99% of one or more carriers. Compositions for parenteral administration typically include about 0.1% to about 10% of an active compound and about 90% to about 99.9% of a carrier including a diluent and a solvent.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. In some embodiments, peroral liquid compositions include one or more ingredients selected from colorants, flavors, and sweeteners.

Other compositions useful for attaining systemic delivery of the disclosed agents include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions can further include lubricants, colorants, flavors, sweeteners, antioxidants, and/or glidants.

The amount of the carrier employed in conjunction with a disclosed agent(s) is sufficient to provide a practical quantity of composition for administration per unit dose of the agent(s). Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

Pharmaceutical excipients useful for the compositions as described herein include, but are not limited to: acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); alkalizing agents (ammonia solution, ammonium carbonate, diethanolamine, diisopropanolamine, potassium hydroxide, sodium bicarbonate, sodium borate, sodium carbonate, sodium hydroxide, trolamine); antifoaming agents (dimethicone, simethicone); antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, ascorbic acid, thimerosal, thymol); antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); chelating agents (edetate disodium, ethylenediaminetetraacetic acid and salts, edetic acid); coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, camauba wax, microcrystalline wax, zein); colorants (caramel, red, yellow, black or blends, ferric oxide); complexing agents (ethylenediaminetetraacetic acid and salts (EDTA), edetic acid, gentisic acid ethanolamide, oxyquinoline sulfate); desiccants (calcium chloride, calcium sulfate, silicon dioxide); emulsifying and/or solubilizing agents (acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax); filtering aids (powdered cellulose, purified siliceous earth); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); humectants (glycerol, hexylene glycol, sorbitol); plasticizers (e.g., castor oil, diacetylated monoglycerides, diethyl phthalate, glycerol, mono- and di-acetylated monoglycerides, propylene glycol, triacetin, triethyl citrate); polymers (e.g., cellulose acetate, alkyl celluloses, hydroxyalkyl, acrylic polymers and copolymers); solvents (acetone, alcohol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or viscosity-increasing agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); surfactants (simethicone); tablet binders (acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); tablet disintegrants (alginic acid, microcrystalline cellulose, croscarmellose sodium, crospovidone, polacrilin potassium, sodium starch glycolate, starch, pregelatinized starch); tablet and/or capsule lubricants (calcium stearate, glyceryl behenate, magnesium stearate, light mineral oil, sodium stearyl fumarate, stearic acid, purified stearic acid, talc, hydrogenated vegetable oil, zinc stearate); thickening agents (gelatin having a bloom strength of 50-100); tonicity agent (dextrose, glycerol, mannitol, potassium chloride, sodium chloride); vehicle: flavored and/or sweetened (aromatic elixir, compound benzaldehyde elixir, iso-alcoholic elixir, peppermint water, sorbitol solution, syrup, tolu balsam syrup); vehicle: oleaginous (almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane); vehicle: solid carrier (sugar spheres); vehicle: sterile (bacteriostatic water for injection, bacteriostatic sodium chloride injection); viscosity-increasing (see suspending agent); water repelling agents (cyclomethicone, dimethicone, simethicone); and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol). This list is not meant to be exclusive, but instead merely representative of the classes of excipients and the particular excipients that may be used in oral dosage forms as described herein. See Remington's Essentials of Pharmaceutics, Pharmaceutical Press Publishing Company, London, UK, 1^{st} Edition, 2013, and the Handbook of Pharmaceutical Excipients, 8^{th} Edition, Pharmaceutical Press Publishing Company London, U K, 2017, each of which is incorporated by reference herein for such teachings.

Also described herein are methods for manufacturing a dosage form comprising formulating an agent or composition thereof as described herein comprising sprays, capsules, tablets, elixirs, emulsions, lozenges, suspensions, syrups, pills, lotions, epidermal patches, suppositories, inhalers, or injectables. Any methods known to the art for formulating extracts or active principal ingredients into lotions, soaps, etc. can be utilized. In some embodiments, the agents are formulated as individual, immediate release dosage forms. In some embodiments, the agents are formulated as individual, controlled release dosage forms.

### 4. Kits

Also disclosed herein are kits that can be used for, e.g., treating a cancer. The kit can include a first agent that inhibits cellular nucleus export, a second agent that inhibits Akt, and one or more packages, receptacles, delivery devices, labels, or instructions. The kit can also include compositions of the first agent and the second agent as disclosed herein. The description of the first agent, the second agent, compositions, and pharmaceutical compositions described above may also be applied to the disclosed kits.

In addition, the kit may include a packaging configured to contain the first agent and the second agent. The packaging may be a sealed packaging, such as a sterile sealed packaging. By “sterile” it is meant that there are substantially no microbes (such as fungi, bacteria, viruses, spore forms, etc.). In some embodiments, the packaging may be configured to be sealed, e.g., a water vapor-resistant packaging, optionally under an air-tight and/or vacuum seal.

In some embodiments, the kit includes the first agent and the second agent in separate containers. In some embodiments, the kit includes the first agent and the second agent in separate containers in the packaging.

The kits may further include instructions for using the first agent, the second agent, and compositions thereof. These instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another form for the instructions could be a computer readable medium, e.g., computer-readable memory (e.g., flash memory), etc., on which the information has been recorded or stored. Yet another form for the instructions that may be present is a website address which may be used via the Internet to access the information at a removed site. Any convenient means may be present in the kits.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for treating a cancer in a subject in need thereof, the method comprising administering to the subject an effective amount of a first agent that inhibits cellular nucleus export and an effective amount of a second agent that inhibits Protein kinase B (Akt).
Clause 2. The method of clause 1, wherein the first agent inhibits exportin 1.
Clause 3. The method of clause 1 or 2, wherein the first agent comprises Selinexor, Eltanexor, or a combination thereof.
Clause 4. The method of any one of clauses 1-3, wherein the second agent inhibits Akt via inhibiting a G-protein coupled receptor (GPCR), a phosphoinositide 3-kinase (PI3K), or a combination thereof.
Clause 5. The method of any one of clauses 1-4, wherein the second agent inhibits Akt via inhibiting purinergic receptor P2RY2, phosphoinositide 3-kinase gamma (PI3K γ), or a combination thereof.
Clause 6. The method of any one of clauses 1-5, wherein the second agent comprises MK-2206 2HCl, Perifosine, GSK690693, AZD5363, Ipatasertib, Capivasertib, PF-04691502, AT7867, Tricirbine, CCT128930, A-674563, PHT-427, Miransertib, BAY1125976, Borussertib, Miransertib, Akti-1/2, Uprosertib, Afuresertib, AT13148, Oridonin, Miltefosine, Honokiol, TIC10 Analogue, Urolithin B, Resibufogenin, Cinobufagin, Daphnoretin, Loureirin A, Trigoneline, ML-9 HCl, ABTL-0812, Alobresib, Praeruptorin A, Oroxin B, SC66, Usnic acid, Scutellarin, Astragaloside IV, Deguelin, TIC10, Methyl-Hesperidin, IPI-549, TAS-117, ARQ-751, LY2780301, or a combination thereof.
Clause 7. The method of any one of clauses 1-6, wherein the second agent comprises Ipatasertib.
Clause 8. The method of any one of clauses 1-7, wherein the cancer comprises leukemia, lymphoma, myeloproliferative neoplasms, myelodysplastic syndromes, amyloidosis, Waldenstrom's macroglobulinemia, aplastic anemia, myeloma, or solid cancers.
Clause 9. The method of any one of clauses 1-8, wherein the cancer comprises acute myeloid leukemia (AML).
Clause 10. The method of any one of clauses 1-9, wherein the first agent is administered concurrently with the second agent to the subject.
Clause 11. The method of any one of clauses 1-10, wherein the second agent is administered to the subject after the first agent is administered.
Clause 12. The method of any one of clauses 1-11, wherein the second agent is administered at least 8 hours after the first agent is administered.
Clause 13. The method of any one of clauses 1-12, wherein the effective amount of the first agent comprises about 0.1 mg/kg to about 100 mg/kg, the effective amount of the second agent comprises about 0.1 mg/kg to about 100 mg/kg, or a combination thereof.
Clause 14. The method of any one of clauses 1-13, wherein the first agent and the second agent are administered as a single dosage form.
Clause 15. The method of any one of clauses 1-14, wherein the subject is a mammal.
Clause 16. The method of any one of clauses 1-15, wherein the second agent inhibits a pro-oncogenic effect of the first agent.
Clause 17. The method of any one of clauses 1-16, wherein the method decreases a number of CD45+ cancer cells in the subject compared to an administration of the first agent alone or the second agent alone.
Clause 18. The method of any one of clauses 1-17, where the method increases a time of survival of the subject compared to an administration of the first agent alone, the second agent alone, or cytarabine and doxorubicin.
Clause 19. The method of any one of clauses 1-18, wherein the method decreases a number of leukemia-initiating cells in the subject compared to an administration of cytarabine and doxorubicin.
Clause 20. A composition comprising:
   a first agent that inhibits cellular nucleus export; and
   a second agent that inhibits Akt; and
   one or more pharmaceutically acceptable excipients.
Clause 21. The composition of clause 20, wherein the pharmaceutically acceptable excipients comprise buffering agents, solubilizers, solvents, antimicrobial preservatives, antioxidants, suspension agents, a tablet or capsule diluent, or a tablet disintegrant.
Clause 22. The composition of clause 20 or 21, wherein the first agent inhibits exportin 1.
Clause 23. The composition of any one of clauses 20-22, wherein the second agent inhibits Akt via inhibiting purinergic receptor P2RY2, phosphoinositide 3-kinase gamma (PI3K γ), or a combination thereof.
Clause 24. The composition of any one of clauses 20-23, wherein the composition comprises about 1 mg to about 800 mg of the first agent, comprises about 1 mg to about 800 mg of the second agent, or a combination thereof.
Clause 25. The composition of any one of clauses 20-24, wherein the second agent inhibits a pro-oncogenic effect of the first agent.
Clause 26. A kit comprising:
   a first agent that inhibits cellular nucleus export;
   a second agent that inhibits Akt; and
   one or more packages, receptacles, delivery devices, labels, or instructions for use.

### EXAMPLES

### Example 1

### Materials and Methods

### Cell Lines and Reagents

All cell lines were maintained in a humidified incubator at 37° C. with 5% CO₂. OCI-AML2, MOLM13, MV4; 11, HL-60 and OCI-AML3 cells were cultured in RPMI-1640 medium with 10% fetal bovine serum (FBS) and 1% penicillin/streptomycin. 293FT cells were cultured in DMEM high glucose medium with 10% FBS, 1% penicillin/streptomycin, 1% sodium pyruvate, 1% non-essential amino acids, and 1% GlutaMax. All cell lines were purchased from American Type Culture Collection (ATCC) or Duke University Cell Culture Facility (CCF). Drugs were purchased from ApexBio (MK-2206, BYL-719), Tocris (AR-C 118925XX), Sigma-Aldrich (Pertussis toxin) and SelleckChem (Selinexor, IPI-549, GDC0068, GSK690693).

### Short-Term Drug Sensitivity Assay (GI50).

Short-term cell viability assays were conducted. Briefly, AML cells were seeded at a density of 7,500 cells/well, treated with vehicle (DMSO) or a 10-fold serial dilution of selinexor (individually or combination with fixed-concentration background drug) and assessed for viability after 72-hours using Cell Titer Glo (Promega). The relative cell viability was determined by normalizing the raw luminescence values for each treatment condition to either the DMSO-treated well (selinexor individually) or the background drug only well (selinexor combinations). GI50 values approximate the concentration of selinexor required to inhibit growth of cells by 50%. Values were interpolated from dose-response curves plotted using GraphPad/Prism 8 software.

### Time-to-Progression Assay

Cells were plated into 10 cm plates at a concentration of 1×10⁶ cells per plate, and treated with drug or vehicle. Cells were counted weekly and up to 1×10⁸ were replated into a new 10 cm plate with fresh drug. This was repeated weekly for a total of 8 weeks. Weekly growth rates (μ) were calculated from the number of cells plated the prior week (NO) and the number counted the current week (N) using the formula ln(N)=ln(N0)+μ*t; where t is elapsed time in hours. These growth rates were used to extrapolate the weekly virtual cell number.

### Western Immunoblotting: Immunoblotting

Protein lysates were prepared with 1×CST Lysis Buffer (CST #9803) supplemented with 1× cOmplete protease inhibitor cocktail (Roche #04693124001) and 1× PhosSTOP phosphatase inhibitor (Roche #04906837001). Crude lysates were rotated for 15 minutes with lysis buffer, cleared by centrifugation at 13,000 rpm for 10 minutes at 4° C. and normalized by total protein content using standard Bradford analysis. Membranes were probed with primary antibodies β-actin (13E5) (CST #4970 diluted 1:5000 in 5% BSA), p-AKT T308 (244F9) (CST #4056 diluted 1:1000 in 5% BSA), p-AKT S473 (D9E) (CST #4060 diluted 1:1000 in 5% BSA), T-AKT (C67E7) (CST #4691 diluted 1:3000 in 5% BSA), p-GSK3B S9 (D85E12) (CST #5558 diluted 1:1000 in 5% BSA), p-BAD S136 (D25H8) (CST #4366 diluted 1:1000 in 5% BSA), CRM1 (C-1) (sc #74454 diluted 1:100 in 5% BSA), cleaved-PARP (D64E10) (CST #5625 diluted 1:1000 in 5% BSA), cleaved-Caspase3 D175 (CST #9661 diluted 1:500 in 5% BSA), p110-g (D55D5) (CST #5405 diluted 1:1000 in 5% BSA), p110α (C73F8) (CST #4249 diluted 1:1000 in 5% BSA), p110β (C33D4) (CST #3011 diluted 1:1000 in 5% BSA), p1108 (D1Q7R) (CST #34050 diluted 1:1000 in 5% BSA) or p101 (D32A5) (CST #5569 diluted 1:1000 in 5% BSA) overnight (16 hours). Following incubation with HRP-conjugated secondary antibody, blots were developed with SuperSignal West Pico PLUS Chemiluminescent Substrate (ThermoFisher) or ECL Western Blotting Substrate (ThermoFisher).

### Ras-GTP Immunoprecipitation

Active GTP bound Ras was pulled down and detected using the Active Ras Pull-Down and Detection Kit (Thermo Scientific, #16117) according to manufacturer's instruction. At least 10×10⁶ cells were used in pull-down and lysates were normalized to ensure equal protein input across DMSO and selinexor-treated samples within each cell line.

### RT-qPCR Analysis

RNA was isolated from whole cells with the RNEasy Mini kit (Qiagen). cDNA was reverse transcribed from total RNA samples using iScript cDNA Synthesis Kit with 1 μg of RNA template. qRT-PCR was carried out using iQ SYBR Green Supermix and a CFX384 Touch Real-Time PCR Detection System, according to manufacturer instructions. Fold expression was determined by normalizing cycle threshold (Cq) values to ACTB reference gene and normalizing samples to control sample, in accordance with the ΔΔCq method.

### Lentivirus Production

293FT cells were grown to 70-80% confluency in a 10 cM and transfected using Lipofectamine 2000 (Invitrogen), PLUS Reagent (Invitrogen), 8.164 μg of psPAX2, 5.336 μg of pVSVg, and 10.667 μg of plasmid DNA diluted in Opti-MEM according to manufacturer's instructions. Briefly, psPAX2, pVSVg and plasmid DNA were mixed with 785 μL Opti-MEM. 103.2 UL PLUS Reagent was diluted in 785 μL Opti-MEM and gently pipetted onto DNA mixture. After a 5 minute R.T. incubation, 94.6 μL Lipofectamine 2000 in 1.570 mL Opti-MEM was gently pipetted onto DNA/PLUS Reagent mixture. After another 5 minute R.T. incubation, entire mixture was added dropwise to 10 cM containing 293FT cells. After a 5-hour 37° C. incubation of 293FT cells with transfection mixture, media was aspirated and exchanged for virus harvest media (30% FBS in described 293FT media). After 48 hours at 37° C. media containing virus was harvested, filtered with a 0.45 μm filter and stored at −80° C.

Generation of Stably Expressing Doxycycline-Inducible shRNA Cells

Controlled expression of shRNAs was achieved using a doxycycline-inducible pLKO-Tet-On lentiviral system. Briefly, shRNA sequences were obtained from the LEGACY shRNA inventory, designed as complementary top and bottom oligonucleotide sequences and ordered from IDT. Top and bottom oligos were annealed, ligated with AgeI/EcoRI digested gel-purified pLKO-Tet-On vector, transformed into competent One Shot Stabl3 E. coli cells (Invitrogen #C737303) and spread onto LB/Amp plates. Individual colonies were selected and plasmid DNA was purified (Qiagen) and sequence validated. Lentivirus was generated and cells were transduced as described. Following two days of puromycin selection, shRNA containing cells were treated with doxycycline (75 ng/ml) for 12-72 hours to determine kinetics and efficiency of gene knockdown.

### CRISPR/Cas9 Selinexor Sensitizer Screen

Custom sgRNA library generation: Custom sgRNA library was designed, cloned and amplified. Briefly, the library includes 12,000 sgRNAs targeting 2390 genes (5 sgRNAs per gene) and 50 non-targeting controls. Each unique 20 base pair sgRNA was appended/prepended and synthesized as an oligo pool by Custom Array Inc. The pooled inserts were PCR amplified using NEB Phusion Hotstart enzyme mix and cleaned up with Axygen magnetic PCR beads (Fisher Scientific). Gibson assembly was performed using 100 ng of FastDigest BsmBI digested lentiCRISPRv2 (Addgene plasmid #52961), 40 ng of prepped sgRNA insert and 10 μL of Gibson assembly master mix (NEB). 1 μL of the Gibson reaction product was transformed into electrocompetent cells (E. coli 10G ELITE, Lucigen #60052-2), spread onto LB-ampicillin plates and incubated at 37° C. for 16 hours. Colonies were counted to ensure >40× coverage of library, scraped and plasmid DNA was isolated using a Maxiprep kit (Qiagen).

Individual sgNT and sgXPO1 oligonucleotides were prepared, cleaned-up, cloned, transformed, isolated, and sequence validated in an analogous manner.

### Pooled CRISPR Screening

Virus production, titering and transduction was performed as previously described. In short, viral titer (Infectious Units (IFU)/mL) was found by transducing OCI-AML2 cells with a 1:3 dilution series of library virus, selecting cells with puromycin for 2 days and determining multiplicity of infection (MOI) from % of cells infected. Viral titer is equal to (#cells seeded*MOI*Virus dilution factor)/(Volume of virus added to each well). OCI-AML2 cells were transduced at an MOI of 0.2 at 1000× coverage of the library and puromycin selected for 7 days prior to dividing into selinexor-treated versus vehicle-treated populations. For the selinexor sensitizer screen, selinexor was dosed at 100 nM, a concentration that yielded sufficient selective pressure without excessive cell death (approximately the GI50 concentration) over the two-week screen period. Each drug/vehicle condition was conducted in biologically independent replicate and carried at >1000× coverage for 2 weeks. Genomic DNA was extracted using the DNeasy Blood & Tissue Kit (Qiagen) from 25×10⁶ cell samples taken prior to dividing cells into treatment conditions (time zero) and after completion of the two-week screen. Amplification of the sgRNA barcodes and indexing of each sample was performed via 2-step PCR as previously described.

### Screen Processing and Analysis

To determine differences in sgRNA composition between samples, deep sequencing was performed by Hudson Alpha Institute for Biotechnology using the Illumina Nextseq platform (single-ended 75 base pair reads). As previously described, barcoded reads were converted to guide-level counts and the fractional representation (FR) of each sgRNA construct was found by dividing the count of each sgRNA in a sample by the sum of all sgRNA counts in that sample. The selinexor construct-level depletion score was found by comparing the 2-week drug-treated population to the 2-week vehicle-treated population (Selinexortime=2 weeks/DMSOtime=2 weeks). The construct-level depletion scores were collapsed to gene-level depletion scores by taking the average depletion score across the 5 sgRNA constructs. All depletion/enrichment effects are reported as log 2 ratios. All described manipulations were performed in R.

The results of this sensitizer screen were included in a larger effort, although a full dissection of selinexor's sensitizer interactions has been reserved for this study.

### Reverse-Phase Protein Array (RPPA) Analysis Sample Preparation

OCI-AML2 cells were treated with DMSO or selinexor for 3, 24 and 48-hours at which point 3×10⁶ cells were pelleted and frozen at −80° C. RPPA analysis was performed as previously described. All samples were conducted in biological triplicate and normalized to DMSO control at each respective timepoint.

p-AKT T308 FACS-Based CRISPR/Cas9 Screen in Selinexor Treated OCI-AML2 Cells sgRNA Library Amplification

The Toronto Knockout CRISPR Library—Version 3 (TKOv3) was obtained from Addgene (Pooled Libraries #90294, #125517) and amplified according to provided published protocol. The TKOv3 library contains 70,948 sgRNAs targeting 18,053 protein coding genes (4 sgRNAs targeting each gene) and 142 non-targeting control sgRNAs against LacZ, EGFP and luciferase (total library size is 71,090 sgRNAs). Briefly, TKOv3 pooled plasmid library DNA was diluted 1:10 in TE and electroporated into Endura electrocompetent cells (Lucigen, #60242). A total of four electroporations was performed to ensure coverage of >25× of the library. The library plasmid pool was purified using a Maxiprep kit (Qiagen) and virus was produced and titered in an identical manner as described for the CRISPR/Cas9 selinexor sensitizer screen. OCI-AML2 cells were transduced at 1000× coverage of the library (400×10⁶ cells infected, 72×10⁶ cells transduced) and cultured at a minimum of 1000× coverage.

Fixation and Intracellular p-AKT T308 Staining

Ten days after transduction, TKOv3 library transduced OCI-AML2 cells were treated with DMSO or 200 nM selinexor. After 48-hours, cells were pelleted at 1200 rpm, washed with 1×PBS and fixed/permeabilized using the Thermo IC Fixation Kit. Fixation, permeabilization and staining was performed according to manufacturer's instruction with slight modification. Protocol was performed in 15 mL falcon tubes with 35×10⁶ cells per tube and IC Fixation Kit buffers were scaled accordingly. Cells were fixed at R.T. for 15 minutes with IC Fixation buffer (decreased fixation time increased antibody staining strength and specificity). Cells were washed with 1× Permeabilization Buffer followed by wash with FACS buffer (1×PBS, 2% FBS, 0.1% Sodium Azide, 2 mM EDTA). Cells were stained in 1× Permeabilzation Buffer with a 1:200 dilution of p-AKT T308 (244F9) (CST #4056) primary antibody (1 mL of Permeabilzation Buffer/primary antibody solution per 35×10⁶ cells) at R.T. for 2 hours with gentle rocking. Cells were again washed with 1× Permeabilization Buffer followed by FACS buffer and stained in 1× Permeabilzation Buffer with 1:200 dilution of Alexa Fluor 488 Conjugate (CST #4412) fluorophore-conjugated anti-rabbit secondary antibody at R.T. for 1 hour with gentle rocking. Cells were washed with 1× Permeabilization Buffer followed by FACS buffer and resuspended at 25×10⁶ cells/mL in FACS buffer in preparation for FACS analysis. All centrifugations performed at 700×g unless otherwise indicated.

### FACS Analysis and Sorting

To achieve sufficient coverage of the library in the sorted cell populations, 144×10⁶ (2000× coverage) selinexor-treated, fixed and stained cells were sorted in each replicate. This ensured that both the top and bottom sort populations retained at least 200× coverage of the library. Cells were strained with a 0.3 μm filter, FACS analyzed and sorted using the Astrious Cell Sorter (Beckman Coulter). Cells were gated for live cells based on FSC/SSC and singlets based on FSC. The bottom 18% and top 10% p-AKT T308 expressing cells were collected into 1×PBS in separate collection tubes. The bottom 18% gate represents selinexor-treated cells that were unable to activate AKT relative to DMSO-treated cells. To prevent the fixed cells from sticking to the collection tubes, 2% FBS was spiked into sorted cell populations. Cells were distributed into 1.5×10⁶ cell aliquots, pelleted at 700 g, followed by genomic DNA extraction using Arcturus PicoPure DNA Extraction Kit (ThermoFisher #KIT0103) according to manufacturer's instructions. Amplification of the sgRNA barcodes and indexing of each sample was performed via 2-step PCR as previously described.

### Screen Processing and Analysis

To determine the sgRNA composition in the bottom and top-sorted cell populations, deep sequencing was performed by the Duke Sequencing and Genomic Technologies core using the Illumina NovaSeq 6000 platform (single-ended 75 base pair reads). Barcoded reads were converted to fractional representation as described in analysis of CRISPR/Cas9 selinexor sensitizer screen. Construct-level scores were found by comparing the fractional representation of each sgRNA in the bottom sort population to that of the top sort population (SelinexorBottom sort/SelinexorTop sort). The construct-level scores were collapsed to a gene-level FACS screen gene score (FSGS) by taking the average score across the 4 sgRNA constructs. All FSGSs are reported as log 2 ratios. All described manipulations were performed in R.

### RNA-Seq Gene Expression Analysis

OCI-AML2 and MOLM-13 cells were treated for 36 hours with vehicle or selinexor (200 nM for OCI-AML2 and 75 nM for MOLM-13) in biologically independent triplicate. RNA was isolated from whole cells with the RNEasy Mini kit (Qiagen) and sent for paired-end RNA-sequencing by Novogene. Mapped reads were analyzed using DEseq and assessed for differential expression in selinexor versus vehicle-treated conditions.

Single-Sample GSEA (ssGSEA)

ssGSEA is an unsupervised gene enrichment method that was here used to calculate separate enrichment scores (ES) for each pairing of a sample whose transcriptomic data was available from TCGA-LAML (n=198 AML samples) or GSE14468 (n=526 AML samples) and either the AKT/RAS/MAPK-related gene sets (queried from the MSigDB database), or the common selinexor-induced gene signature derived from MOLM-13 and OCI-AML2 AML cells treated with DMSO or Selinexor. The AKT/RAS/MAPK-related gene sets follow the annotation format of “insertname”.V1_UP or “insertname”.V1_DN. The chemical or genetic perturbation is indicated in the “insertname” descriptor. Gene sets upregulated or downregulated in response to “insertname” perturbation are labeled with suffixes _UP or _DN, respectively. To generate the selinexor signature, the top 300 differentially up and downregulated genes between DMSO and Selinexor conditions (defined based on the lowest adjusted p-value and log 2 fold change) were derived from each cell lines, and at the intersection of these two gene lists, a common signature was identified for 166 and 122 genes that were up and downregulated, respectively, in both AML cell lines. A positive ES denotes a significant overlap of the signature gene set with groups of genes at the top of the ranked list, whereas a negative ES denotes a significant overlap of the signature gene set with groups of genes at the bottom of the ranked list. ES scores which were obtained from each patient sample interrogated with all gene sets, were further z-score normalized. A ssGSEA z-score cutoff 1 was used to assign a tumor sample as highly enriched in any of the gene signatures. Only patient samples exhibiting a high (ES z-score>1) or low (ES z-score<−1) selinexor signature were taken into account to join in a similar cluster the gene sets whose profile of enrichment is the most similar between primary samples.

### P2RY2 Expression Patient Stratification

Gene expression data of each AML patient from both cohorts were z-score normalized and high versus low P2RY2 levels were evaluated based on the absolute z-score cut-off of 0.75. On versus off selinexor signatures were assigned for each patient based on the ES z-score>1 or <−1, respectively. The significance of the differences between the proportions of each subgroup of patients was evaluated by applying the two tailed Fisher's Exact Test implemented in the function fisher.test (library stats, R 2.14, cran.r-project.org/).

### Gene Ontology Analysis

Gene Ontology analysis was performed on genes enriched in the bottom sort versus top sort of p-AKT T308 FACS screen (FSGS>1.5) using Enrichr web-based tool (https://amp.pharm.mssm.edu/Enrichr/). Gene Ontology on selinexor sensitizer and resister genes was performed independently on genes with a depletion score of <−0.75 and >0.75, respectively.

### Patient Samples

Synergy assessment: Having informed patient consent under a St Louis Hospital Internal Review Board approved protocol, patient blood or bone marrow samples were collected and subjected to Ficoll (Ficoll-Paque PLUS 17-1440-02 GE Healthcare) gradient centrifugation to collect mononucleated live cells. Red blood cells were lysed (Red Blood Cell Lysing Buffer Hybri-Max R7757 Sigma Life Science) and the blasts were resuspended in patient medium (RPMI 10% FBS 1% Pen/Strept with the cytokines TPO, EPO, SCF, FLT3, IL3, IL6, G-CSF, GM-CSF). Cells were seeded into 384 well plates at a density of 5000 cells per well and treated in quadruplicate with top doses of 1 μM for selinexor and 50 μM for ipatasertib with 1:2 dilutions between doses. After 120 hours, CellTiter-Glo® Luminescent Cell Viability Assay was used as a readout of viability. The presence or absence of synergy across the matrix was assessed by calculating the average Bliss score across all doses using synergyfinder. Negative scores indicate antagonism while positive scores indicate synergy. For the purpose of this study, combinations where the dose of either drug alone produced greater than 90% loss of viability, thus precluding observable synergy, were excluded; Bliss cutoff of positive one was used to denote the presence of strong synergy.

### Patient Profiling

As part of the standard AML patient profiling carried out at St Louis Hospital, clinical, cytogenetic, and genetic characteristics of each sample are collected from medical charts and electronic medical records in an identity-blinded fashion, in accordance with the ethical guidelines of the Declaration of Helsinki. Also, as part of the standard profiling, cytogenetic analyses were carried out using conventional karyotyping and additional FISH studies guided by the karyotype, while genetic profiling consists of fragment analysis for NPM1, FLT3, and IDH1/2 mutational status and by targeted-sequencing of a panel of 85 genes recurrently found mutated in AML at >500× coverage (Agilent SureSelect, Illumina).

### Methylcellulose Assay

For patient samples 11 and 17, additional primary AML cell aliquots were obtained from the bone marrow aspirate, seeded in methylcellulose-based medium MethoCult H4435 (Stem Cell Technologies) at a concentration of 20×10³ cells/plate in triplicate, and treated with the indicated concentrations of Selinexor, Ipatasertib, or the combination of both compounds. Plates were scored for colony formation 14 days later with MTT staining. In vivo transplantation: OCI-AML2 cell line xenograft: The Duke University Institutional Animal Care & Use Committee (IACUC) reviewed and approved the cell line xenograft transplantation and treatment protocol described in this study. OCI-AML2 cells were IMPACT tested and confirmed mycoplasma negative prior to engraftment. Approximately 1×10⁶ luciferase-expressing OCI-AML2 cells suspended in 0.1 mL sterile 1×PBS were tail vein injected into 5-6 week old male NSG mice. Two weeks after injection, mice were assessed for successful engraftment by IVIS bioluminescence imaging and analysis using Living Image software. Mice were sorted by bioluminescence, treated M/W/F with selinexor (10 mg/kg) by oral gavage, ipatasertib (75 mg/kg) by oral gavage, or both selinexor and ipatasertib. Drugs were formulated in OraPlus suspending vehicle. Mice were monitored regularly for signs of distress, such as weight loss >15%, ruffled coat, lethargy, or bruising. Drug treatments and routine monitoring continued for 6 weeks or until a humane endpoint was reached.

### PDX Models

Primary patient AML blasts were collected from bone marrow aspirates after obtaining informed patient consent under a St Louis Hospital Internal Review Board approved protocol. Mononuclear cells were isolated using Ficoll-Paque Plus (Amersham Biosciences) and red blood cells were lysed before flow cytometry analysis. These cells were maintained in StemSpan SFEM (StemCell Technologies, catalog no. 09650) medium supplemented with 20 ng/ml IL-3 (Peprotech, catalog no. 200-03), 20 ng/ml IL-6 (Peprotech, catalog no. 200-06), 20 ng/ml GM-CSF (Peprotech, catalog no. 300-03), 100 ng/ml FLT3-ligand (Peprotech, catalog no. 300-19) and 100 ng/mL SCF (Peprotech, catalog no. 300-07) before injection into NOD.Cg-Prkdcscid Il2rgtm1Sug Tg(SV40/HTLV-IL-3, CSF2)10-7Jic/JicTac (huNOG-EXL) mice purchased from Taconic. Sample size was chosen in light of the fact that these in vivo models were historically highly penetrant and consistent. Animals were excluded from the study if any signs of distress were observed without clinical signs of leukemia: that is, absence of leukemic blasts in bone marrow, spleen and blood. None of the animals were excluded on the basis of these criteria. Blinded observers visually inspected mice for obvious signs of distress, such as loss of appetite, hunched posture and lethargy. Approximately 0.5×10⁶ cells were tail-vein-injected as a secondary transplant into sublethally irradiated (125 cGy) 6-8-week-old male huNOG-EXL mice. Twelve days after injection, mice were assessed for successful engraftment: peripheral blood samples and bone marrow biopsies were resuspended in PBS, 0.5% BSA, 2 mM EDTA prior to staining with an anti-human PE-Cy7-coupled CD45 (hCD45) antibody. Cells were then washed three times in PBS 2 mM EDTA and the proportion of hCD45-positive cells was assessed using a FACScanto II. Following confirmed engraftment, mice were randomized and treated every other day for one week either by oral gavage with 65 mg kg⁻¹ ipatasertib (OraPlus) or 15 mg kg⁻¹ selinexor (OraPlus) or with these two drugs used combined as indicated in the figures. After one week, mice were sacrificed and bone marrow was harvested from legs and backbone to analyze the proportion of leukemic cells in each group. Samples were washed once in PBS and resuspended in 0.5% BSA, 2 mM EDTA-PBS before staining with either APC-conjugated anti-human CD45 (BioLegend, catalog no. 368512, 3:100) antibody and flow cytometry analysis.

### PDX Patient Characteristics

PDX1 was taken from a 69-year-old female who was diagnosed with secondary AML with MDS-related changes; patient was previously treated with mitoxantrone/etoposide/cytarabine+lenalidomide; genetic profiling revealed mutations in CEBPA/ASXL1/RUNX1/EZH2/JAK2/TET2. PDX2 was taken from a 44-year-old female who was diagnosed with relapsed AML; patient was previously treated with allogeneic HSCT/sorafenib/hydroxyurea/decitabine; genetic profiling revealed mutations in FLT3-ITD/NPM1/DNMT3A/IDH1.

### MLL-AF9 Model

Bone marrow from 6-week-old C57BL/6 male donor mice (The Jackson Laboratory) injected with MLL-AF9 dsRed+ cells into the tail vein was harvested from legs and backbone. Approximately 0.1×106 dsRed+ sorted cells were tail-vein-injected as a secondary transplant into sublethally irradiated (350 cGy) 6-8-week-old male C57BL/6 mice. Ten days after injection, mice were randomized and treated every other day for 5 or 10 days either by oral gavage with 65 mg kg⁻¹ ipatasertib (OraPlus) or 15 mg kg⁻¹ selinexor (OraPlus) or with these two drugs combined as indicated in the figures. Chemotherapeutic agents, cytarabine and doxorubicin were resuspended in HBSS and both delivered intraperitoneally at 1 mg/kg and 100 mg/kg respectively on days 1 to 3 and cytarabine alone on days 4 and 5. Bone marrow biopsies were performed on anesthetized animals 24 hours after the end of the treatment, and biopsies were washed once in PBS and resuspended in 0.5% BSA, 2 mM EDTA-PBS before flow cytometry analysis. Upon disease relapse, mice were sacrificed, and bone marrow and spleen were collected, washed with PBS, and resuspended in 0.5% BSA, 2 mM EDTA-PBS before flow cytometry analysis.

### MLL-AF9 Limiting Dilution Assay

20×106 viable MLL-AF9-positive leukemic cells were harvested and sorted from sick mice treated with either the combination of Selinexor (15 mg/kg) and Ipatasertib (65 mg/kg) or the combination of the chemotherapeutic agents Cytarabine (100 mg/kg) and Doxorubicin (1 mg/kg) for 16 hrs. The sorted MLL-AF9 cells were then serially diluted to obtain the appropriate cell concentrations prior to reinjection into sublethally-irradiated secondary recipient mice (either 45,000, 15,000, 5,000, or 1,667 cells per mouse in a total 5 mice per group). Demised mice were then counted and limiting dilution analyses were carried out using the Extreme Limiting Dilution Analysis (ELDA) function of the ‘StatMod’ package (bioinf.wehi.edu.au/software/elda/index.html). Leukemia-Initiating Cell (LIC) frequencies between groups of secondary recipient animals were compared using the likelihood ratio chi-squared test.

The French National Committee on Animal Care reviewed and approved all experiments using the PDX and MLL-AF9 mouse models described in this study.

### Example 2

### Suppressing Drug-Induced Activation of P2RY2/AKT Signaling Potentiates the Therapeutic Effect of Nuclear Export Inhibition in AML

### Parallel Profiling Nominates AKT Activation as a Selinexor-Induced Cell-Beneficial Effect

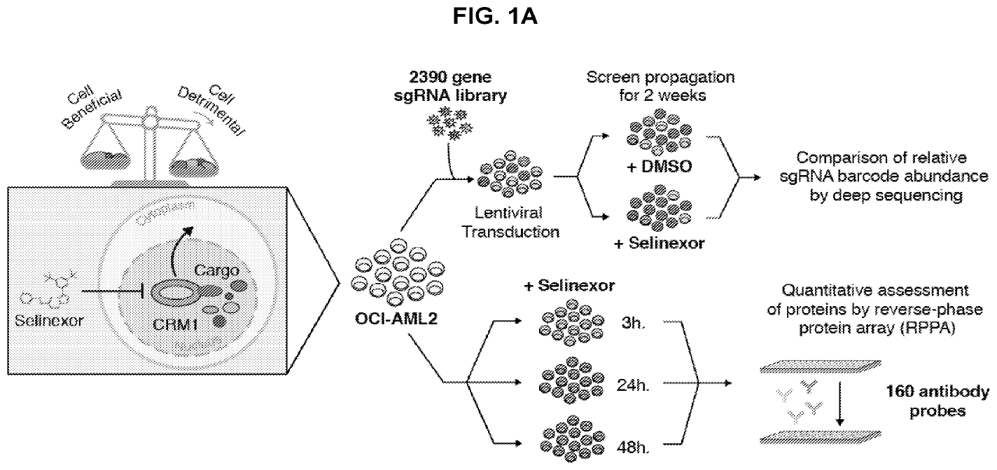

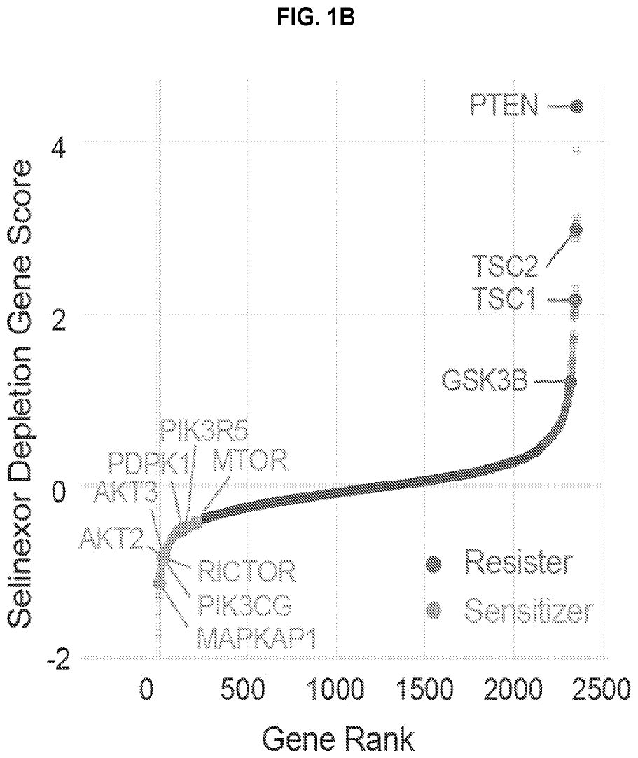

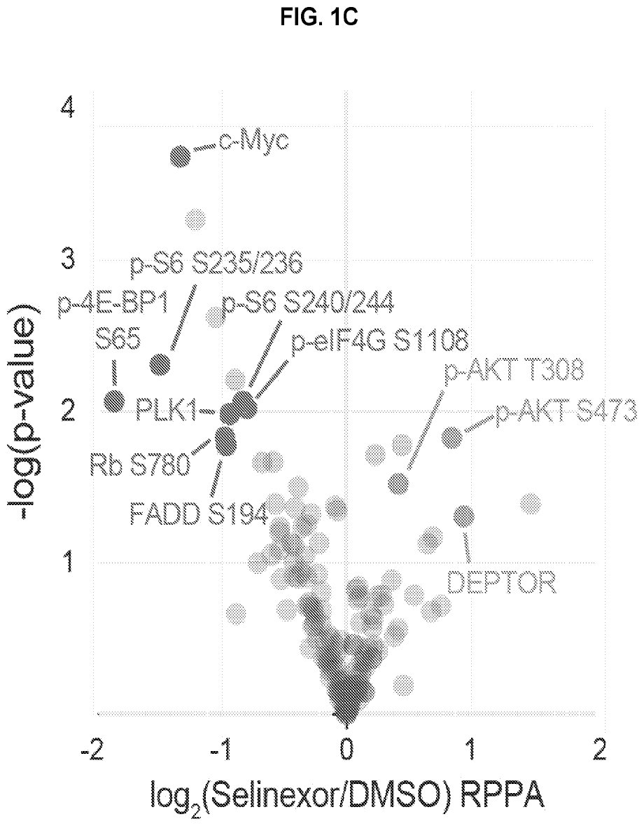

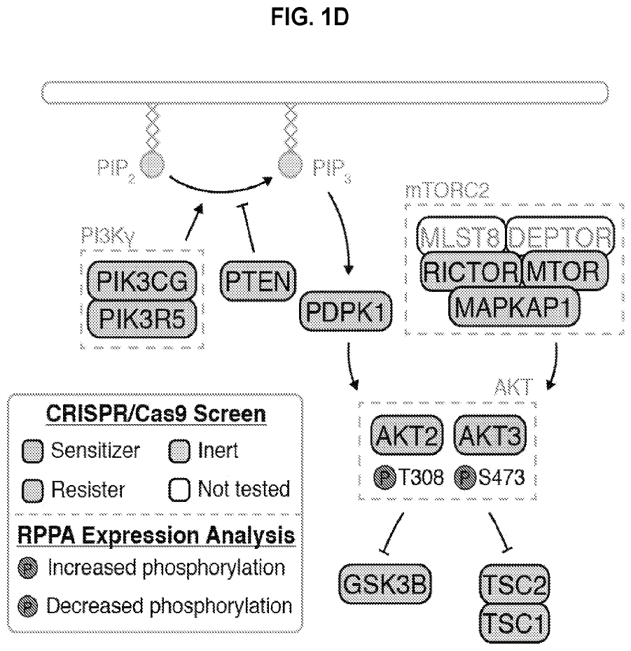

To identify direct, cell-beneficial sequelae of selinexor treatment that could be targeted for therapeutic benefit, biological pathways were searched that satisfied two requirements: (1) treatment of AML cells with selinexor affected the pathway, and (2) genetic or pharmacological modulation of the pathway sensitized AML cells to selinexor treatment. Phenotypic screens were designed to address each of these requirements (FIG. 1A).

To start, a CRISPR/Cas9-based loss-of-function screen was used to identify genetic sensitizers to selinexor. In this assay, OCI-AML2 cells were transduced with a CRISPR/Cas9-knockout library and cultured in the presence or absence of selinexor for two weeks. Samples from zero- and two-week time points were deconvoluted using deep sequencing to identify potential genetic sensitizers to selinexor, genes whose genetic ablation reduced their relative representation within the population of selinexor-treated cells. Because of the interest in genetic sensitizers, a library focused on key oncogenic, proliferative, and survival pathways whose aberrant activation may undermine drug response was used. Including controls, this library totaled 11,950 short guide RNA constructs targeting 2390 genes plus 50 non-targeting short guide RNA constructions. To enforce reproducibility, the screen was performed and analyzed in replicate.

The screen identified a number of selinexor sensitivity modifiers. Among resisters, genes whose loss conferred resistance to selinexor and whose representation was therefore enriched in the presence of drug, gene ontology (GO) pathway analysis singled out cell cycle modulators, specifically the tumor suppressors and known CRM1 substrates p21 (encoded by CDKN1A), p27 (encoded by CDKN1B), RB (encoded by RB1), and p53 (encoded by TP53). These findings cohered with selinexor's known ability to induce G1 arrest and apoptosis, given the roles that p21, p27, RB, and p53 play in restricting G1/S progression and the role of p53 in apoptosis, and are consistent with the notion that these tumor suppressors are sequestered in the nucleus following selinexor treatment. p53, in particular, has been reported as an important determinant of therapeutic response to CRM1 inhibition in AML. Relatedly, BRD1, which indirectly affects the cell cycle through transcriptional regulation of CDKN1A and CDK1, likewise scored as a resister. The screen also identified sensitizers, genes whose loss potentiated the effects of selinexor, thus depleting their representation in the presence of drug. Many of these sensitizers collectively suggested that interference with cell cycle progression (CDK2, E2F3) and c-MYC targets (KAT2A, TAF12, RUVBL1, SUPT3H) could sensitize AML cells to selinexor. Here, the cell cycle genes that scored as sensitizers are directionally consistent with those that scored as resisters. RB receives inhibitory phosphorylation from CDK2 and represses E2F3.

The strongest phenotype identified in the loss-of-function CRISPR screen belonged to PTEN, which was enriched more than 16-fold in the selinexor-treated versus control-treated populations, identifying it as a resister (FIG. 1B). PTEN catalyzes the dephosphorylation of PIP3 to PIP2, and plays an inhibitory, tumor suppressive role within the PI3K/AKT pathway, a pathway widely implicated in the establishment and maintenance of cancer. Many other nodes within this pathway scored in the screen; accordingly, both PI3K/AKT signaling and PTEN signaling were identified by GO pathway analysis as strongly enriched signatures among selinexor sensitizers and selinexor resisters, respectively. PIK3CG and PIK3R5 respectively encode p110γ and p101, complementary catalytic and regulatory subunits of PI3-kinase. Together, they oppose the activity of PTEN and are accordingly identified as sensitizers (FIG. 1B). PDPK1, which encodes PDK1, the signaling node canonically located downstream of PI3-kinase, scored as a sensitizer, as did AKT2 and AKT3, encoding isoforms of the PDK1 substrate AKT. AKT also receives activating phosphorylation from mTORC2, a multi-subunit complex whose components, encoded by RICTOR, MTOR, and MAPKAP1, scored as sensitizers (the mTORC2 components encoded by MLST8 and DEPTOR were not targeted by the library) (FIG. 1B). Last, the screen highlighted genes encoding AKT substrates. AKT provides inhibitory phosphorylation to GSK3b, TSC1, and TSC2, all of which scored as resisters (FIG. 1B. These data strongly implicated the PI3K/AKT pathway as a modifier of selinexor sensitivity.

Next, it was sought to identify the signaling nodes activated by selinexor treatment. To do this, a reverse phase protein array (RPPA) was used to analyze AML cells treated with DMSO or selinexor for 48 hours, using a dose of selinexor sufficient to provide an on-target effect but not high enough to impose a meaningful selective pressure. The RPPA platform, which provides quantitative measurements of 160 epitopes and phospho-epitopes representing cellular growth, proliferation, and signaling pathways, identified many drug-induced effects, several of which supported previously described mechanisms of selinexor activity. Again, consistent with reports describing selinexor's ability to induce G1 cell cycle arrest, selinexor treatment suppressed proteins or phosphorylation marks implicated in G1/S progression such as PLK1, phospho-Rb at Ser780, and phospho-FADD at Ser 194 (FIG. 1C). The RPPA also showed evidence of c-MYC suppression following selinexor treatment, consistent with studies that cite c-MYC downregulation as a mechanism for selinexor's ability to blunt DNA damage response (FIG. 1C). In addition to corroborating selinexor's established effects, the RPPA data was able to identify selinexor-induced downregulations of phospho-S6 at Ser235/236 and Ser240/244, phospho-elF4G at Ser1108, and phospho-4E-BP1 at Ser65, which collectively pointed to suppression of mTORC1 signaling (FIG. 1C). This idea has been raised in multiple myeloma and was further supported here by the reciprocal upregulation of DEPTOR, a negative regulator of mTOR. Notably, two of the strongest upregulations identified by RPPA were reserved for AKT (at both its Thr308 and Ser473 sites) (FIG. 1C), aligning the RPPA dataset with the results of the selinexor sensitizer screen. Together, they suggested that selinexor treatment was capable of activating the PI3K/AKT pathway, and that targeting PI3K/AKT pathway nodes could sensitize to selinexor (FIG. 1D).

### Selinexor Treatment Activates PI3K/AKT Signaling

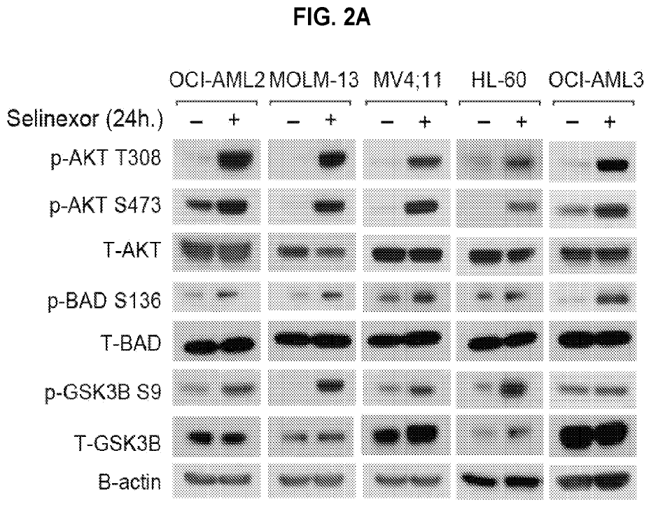

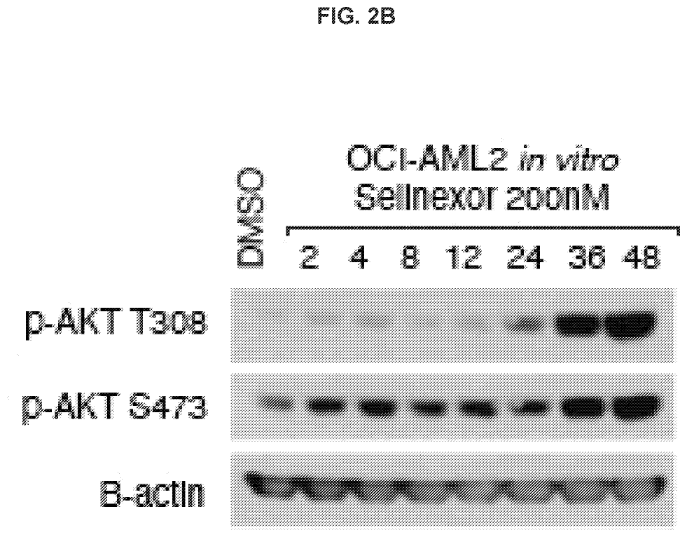

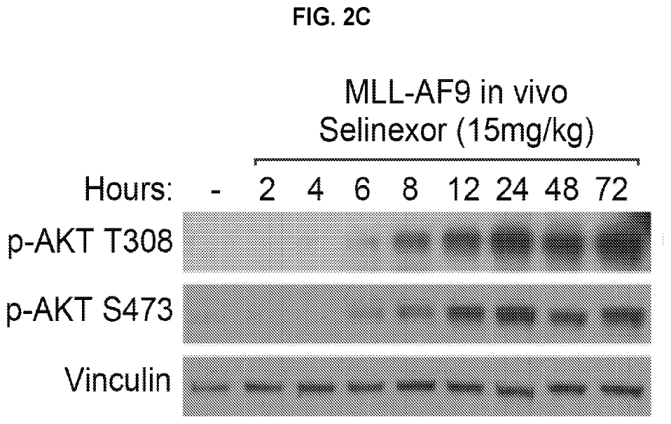

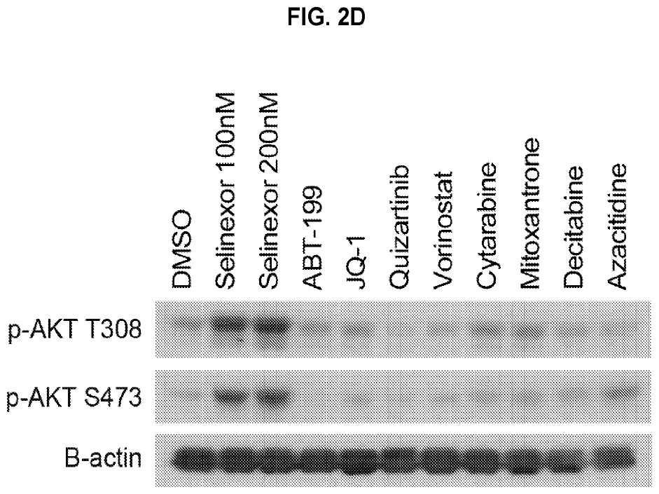

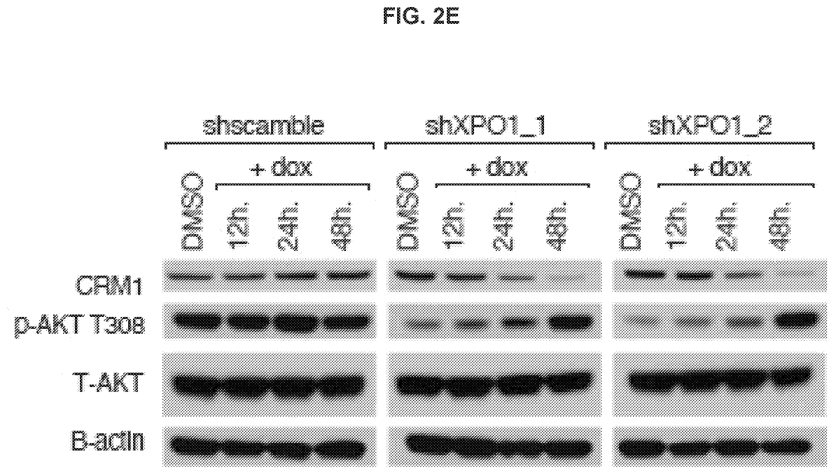

To validate selinexor's ability to activate PI3K/AKT signaling, each of five AML cell lines was treated with selinexor for 24 hours before performing western blot analysis. Across all five cell lines tested, selinexor-treated cells exhibited increased AKT phosphorylation at both Thr308 and Ser473, as well as phosphorylation of GSK3b at Ser9 and BAD at Ser136, well-characterized phosphorylation targets of AKT (FIG. 2A). Additional AKT-dependent phosphorylation marks on PRAS40, FOXO3a, and TSC2 were also assessed in OCI-AML2 cells and found to be increased. This phosphorylation pattern was indicative of activated AKT signaling. To characterize the kinetics of selinexor-induced AKT activation, AML cells were treated with selinexor and collected samples at interval time points up to 48 hours. Across multiple cell lines, activation of AKT signaling was observed starting at 8-24 hours (FIG. 2B). An analogous in vivo experiment was performed using a syngeneic MLL-AF9-driven model of AML, which revealed AKT activation starting 6-8 hours after selinexor treatment (FIG. 2C). The delays between treatment and AKT activation, observed both in vivo and in vitro, were not inconsistent with the presumption that selinexor primarily acts through transcriptional mechanisms. Finally, to assess the exclusivity of these findings for selinexor, cells were treated with a panel of eight additional anti-leukemic drugs for 24 hours. None of the eight drugs were capable of activating AKT, indicating that drug-induced activation of AKT at 24 hours is an uncommon consequence of drug treatment (FIG. 2D).

In order to validate the association between selinexor response and AKT activation, ssGSEA analysis was performed on two published gene expression datasets representing 198 and 526 patients diagnosed with de novo AML. First, commonly-upregulated genes was used from RNAseq analyses of OCI-AML2 and MOLM13 cells treated with selinexor or vehicle to define a selinexor gene signature. This gene signature, along with established gene signatures that represent AKT-induced transcriptional upregulations and downregulations, was used to analyze the two clinical datasets. In both cohorts, consensus hierarchical clustering of samples in the ssGSEA projections revealed that patients who demonstrated enrichment for the selinexor treatment gene signature also demonstrated enrichment for signatures corresponding to AKT-induced transcriptional upregulation while patients who failed to enrich for the selinexor signature instead enriched for signatures corresponding to AKT-induced transcriptional downregulation. Together, these analyses suggest that the transcriptional shift prompted by selinexor treatment is aligned with AKT activation.

Up to this point, all experiments had been performed using selinexor, a well-characterized, first-generation inhibitor of CRM1. To ensure that the effects observed with selinexor were on target, it was next sought to revalidate the claim that selinexor treatment activated AKT using eltanexor, a second-generation inhibitor of CRM1, and genetic knockdown of XPO1, the gene that encodes CRM1. Western blot analysis of eltanexor-treated samples revealed that treatment with eltanexor, similar to selinexor, produced consistent phosphorylation of AKT at Thr308 and Ser473. Next, two doxycycline-inducible shRNAs targeting XPO1 were cloned, both of which were able to induce progressive knockdown of XPO1, and consequently of CRM1, over 48 hours. Over this period, a reciprocal rise in AKT phosphorylation at Thr308 was observed (FIG. 2E). Two sgRNAS were also used to target XPO1 via CRISPR/Cas9, which produced increased phosphorylation at AKT Thr308 and Ser473. These experiments genetically phenocopied the effects of selinexor treatment on AKT signaling, providing functional evidence that the observed effect is the result of on-target CRM1 inhibition by selinexor.

### FACS-Based CRISPR/Cas9 Screening Identifies Genetic Modifiers of Selinexor-Induced AKT Activation

Having established that selinexor treatment activates AKT signaling, attention was turned to the mechanism of AKT activation. It was reasoned that this could be determined by identifying genes that were both necessary for selinexor-induced AKT activation and upregulated in response to selinexor treatment. Finding genes that satisfied these conditions required two separate, unbiased experiments: a functional screen capable of defining the genetic determinants of selinexor-induced AKT activation, and a transcriptomic analysis of selinexor-rversus vehicle-treated cells.

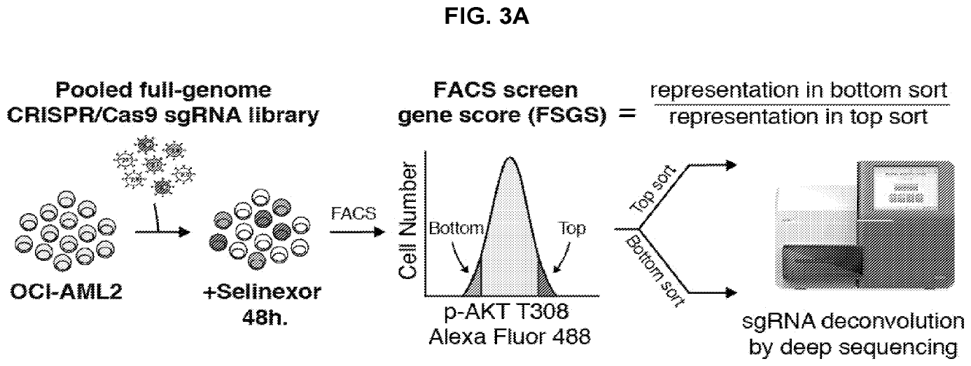

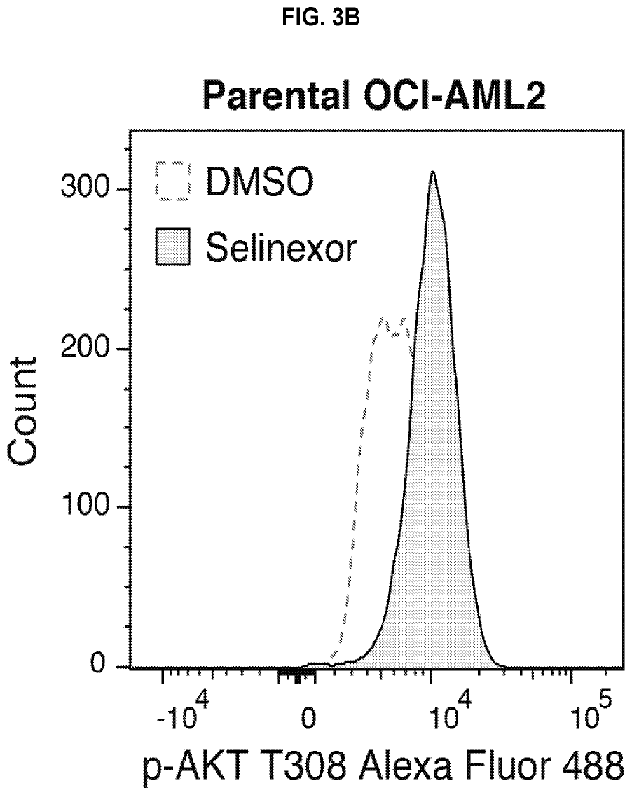

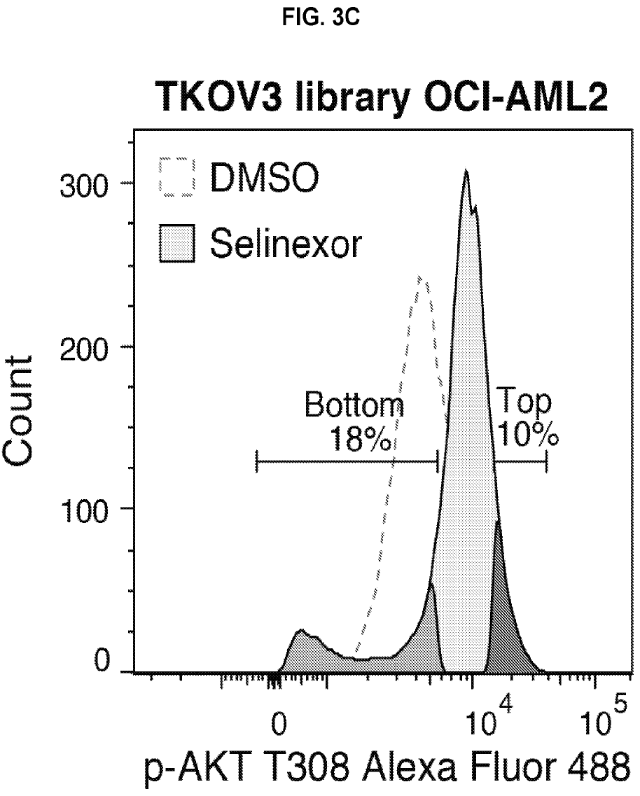

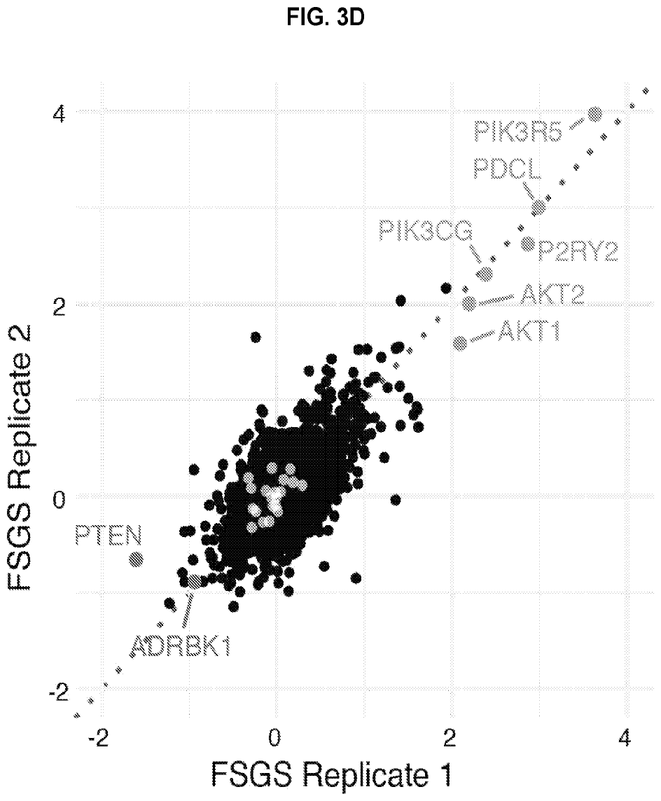

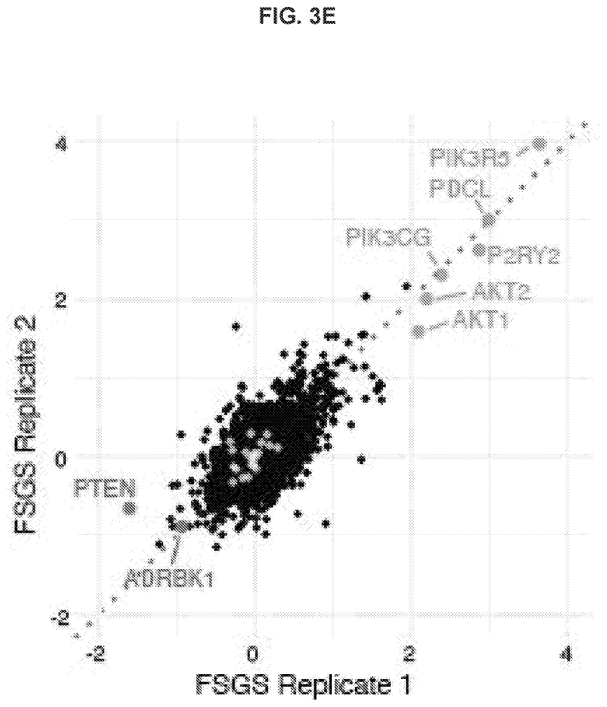

To identify the genes required for selinexor-induced AKT activation, a full-genome loss-of-function CRISPR screen was performed in selinexor-treated cells, using fluorescence-activated cell sorting (FACS) to quantify drug-induced shifts in phosphorylated AKT (FIG. 3A). Phospho-Thr308 was used as the measured phosphoepitope given its primary role in AKT activation and because phosphorylation by PDK1 is a direct product of upstream phosphoinositide mobilization (Ser473 stabilizes Thr308 phosphorylation and is deposited by mTORC2). First, a population of OCI-AML2 cells was stably transduced with a published, full-genome library, then treated library-expressing and library-non-expressing OCI-AML2 cells with selinexor or DMSO. Flow cytometry analysis revealed that, in library-non-expressing cells, selinexor treatment produced a rightward, positive shift in the population distribution of phospho-Thr308 (FIG. 3B). Interestingly, in the library-expressing cells, selinexor treatment produced a bimodal distribution. A larger peak, comprising greater than 90% of the treated population, was right-shifted relative to the control distribution, while a smaller peak was left-shifted relative to the control distribution (FIG. 3C). The presence of bimodality here was important because it provided evidence that the experimental design was sensitive enough to distinguish the subset of genes in the library whose knockout was capable of blocking selinexor-induced AKT activation. From the full distribution, two subpopulations were gated and collected: the top fraction, comprised of cells from the top 10% of the total distribution, and the bottom fraction, the cells whose AKT activation was not induced by selinexor (roughly 18% of the total distribution.) The compositional abundance of sgRNAs in the bottom and top fractions, representing genes whose loss respectively precluded and promoted AKT Thr308 phosphorylation, were deconvoluted through deep sequencing.

In order to stratify genes in the FACS screen by their ability to modulate selinexor-induced AKT activation, it was calculated, for each gene, the ratio of its representation in the bottom fraction over its representation in the top fraction, its ‘FACS screen gene score’, or FSGS. The FSGS is sensitive, as genes that positively affect selinexor-induced AKT activation should be both increased in the numerator (bottom fraction) and suppressed in the denominator (top fraction) of the final quotient (FSGS). Using this approach, a number of candidate positive and negative modifiers of AKT phosphorylation were identified. Among them, AKT1 and AKT2 scored as positive modifiers of AKT activation and PTEN scored as a negative modifier, suggesting that the FACS screen is capable of accurately identifying functional determinants of selinexor-induced AKT activation (FIG. 3D). The FACS screen also identified PIK3CG and PIK3R5 as positive modifiers of AKT activation (FIG. 3D). These themes were independently verified by GO pathway analysis, which pulled out PI3K/AKT-related pathways and also highlighted G protein-coupled receptor (GPCR) signaling (FIG. 3E).

### P2RY2, a Purinergic GPCR, is Upregulated by Selinexor Treatment and Modulates AKT Activation

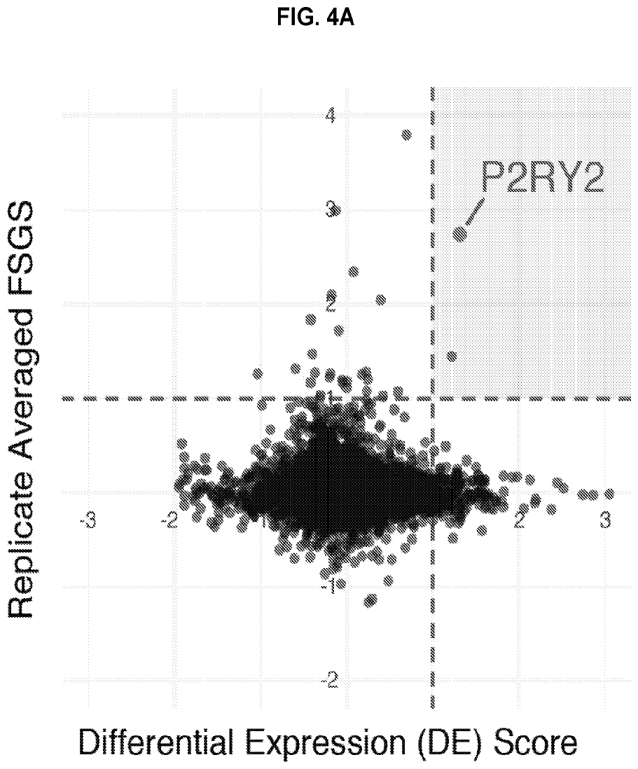

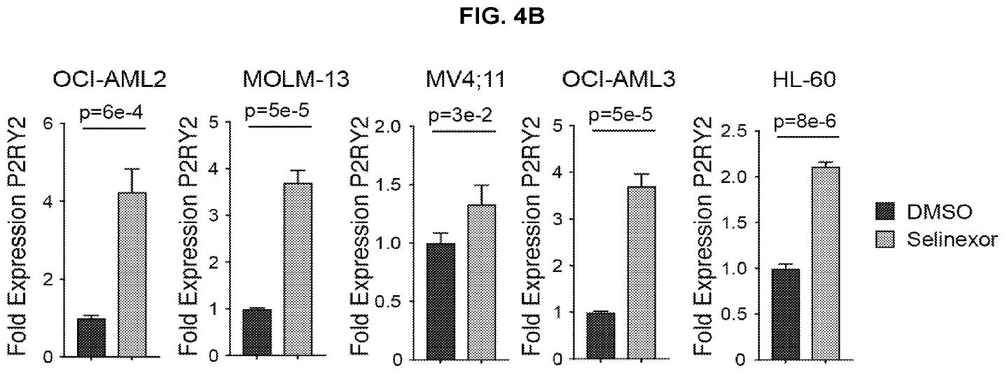

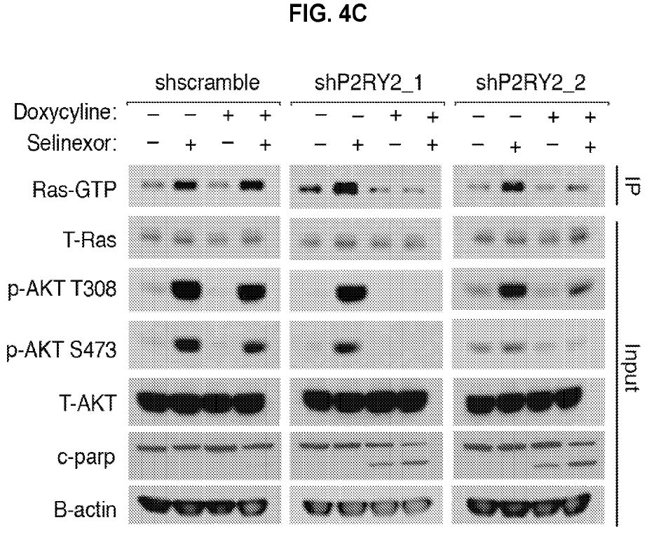

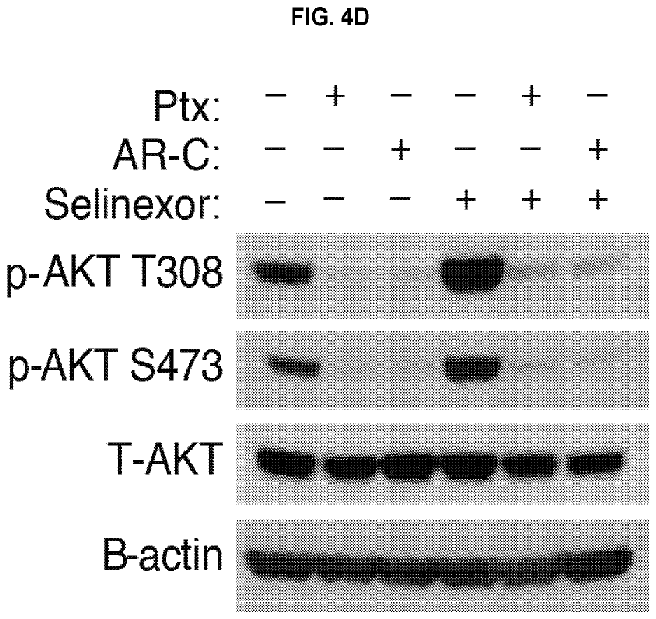

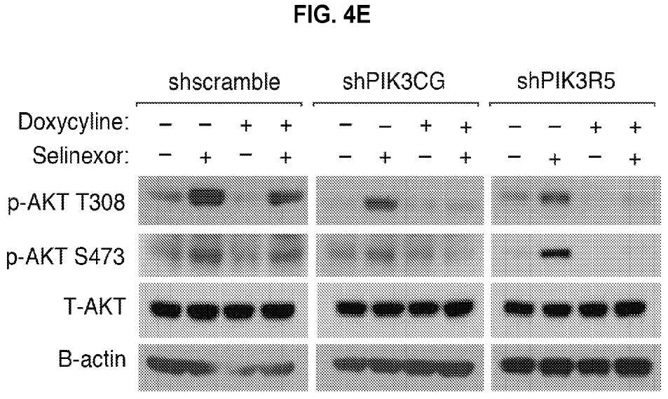

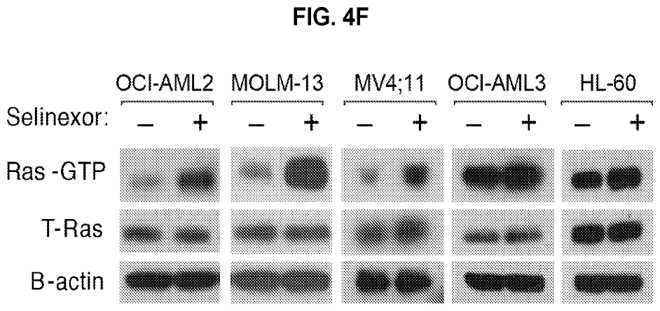

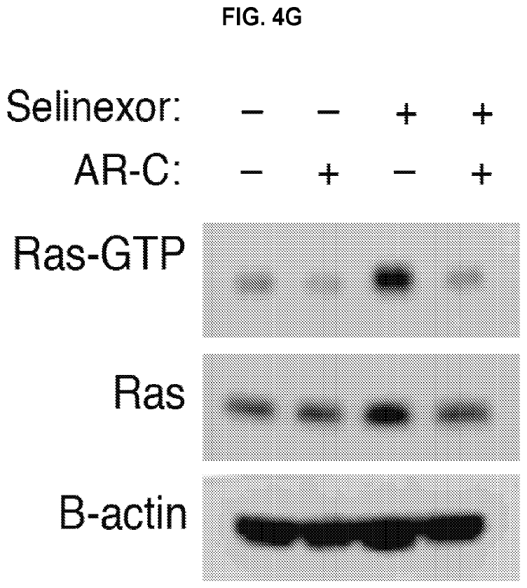

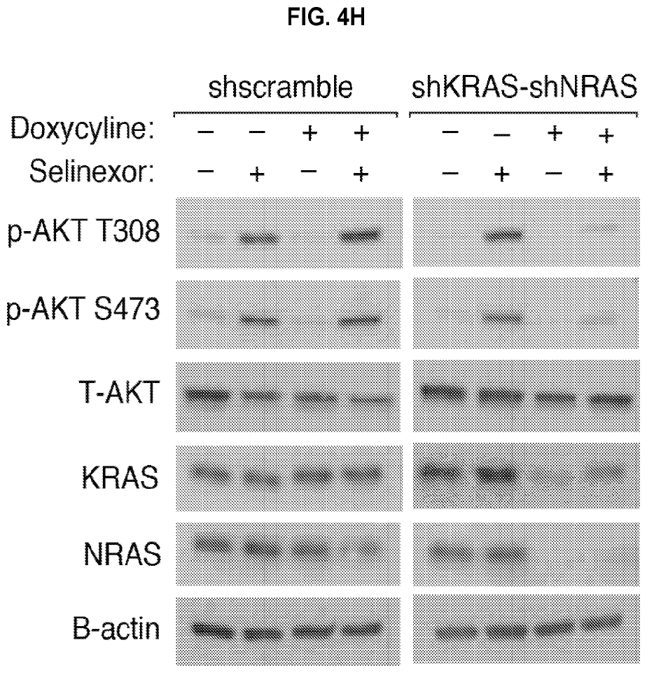

In order to identify genes that were transcriptionally upregulated in response to selinexor treatment, focus was turned to the RNAseq dataset characterizing OCI-AML2 and MOLM13 cells treated with selinexor or vehicle, looking for genes whose expression increased an average of at least two-fold with selinexor-treatment. This analysis yielded a set of 185 genes. Interestingly, one of these genes, P2RY2, was also one of the highest-scoring genes in the FACS screen, suggesting that its selinexor-induced upregulation could be responsible for selinexor-induced activation of AKT (FIG. 4A). First, the upregulation of P2RY2 was validated using qRT-PCR and found that selinexor treatment prompted transcriptional upregulation of P2RY2 starting at 12 and increasing through 48 hours. Further, in each of five AML cell lines, treatment with selinexor increased P2RY2 expression (FIG. 4B). Next, it was reasoned that if transcriptional induction of P2RY2 was indeed a principal determinant of selinexor-induced AKT activation, then baseline overexpression of P2RY2 in AML should predict enrichment for the selinexor gene signature. To test this, the two previously referenced clinical gene expression datasets of de novo AML were reanalyzed, dividing each cohort into groups of high and low P2RY2 expressors. In both datasets, AML samples expressing high P2RY2 were significantly more enriched for the selinexor gene signature than samples expressing low P2RY2. These results independently associate P2RY2 expression with the selinexor-induced gene signature, corroborating the idea that P2RY2 expression, AKT activation, and selinexor treatment may relate meaningfully to one another.

P2RY2 Encodes a Purinergic GPCR that has been Widely Described as an Extracellular ATP and UTP Sensor

The notion that P2RY2 might be responsible for selinexor-induced AKT signaling was intriguing for a few reasons. First, the FACS screen highlighted several GPCR-related genes. The aforementioned PIK3CG and PIK3R5 encode catalytic and regulatory subunits of PI3Kγ. In particular, PIK3R5 encodes p101, a regulatory adaptor of PI3Kγ that facilitates the reception of signaling inputs from GPCRs via Gβγ. PDCL, which encodes a Gβγ modulator, phosducin-like protein, scored as the second-highest positive modifier of AKT activation in the screen (FIG. 3D). ADRBK1, which scored as a negative modifier of AKT activation, encodes GRK2, which phosphorylates phosducin-like protein in a manner that inhibits its capacity to bind Gβγ (FIG. 3D). Second, P2RY2 itself is capable of activating AKT signaling in cancer; it is overexpressed in AML; and it is reportedly transcriptionally upregulated in AML cells when co-cultured with bone marrow adipocytes (representative of an anti-apoptotic microenvironment). Third, as an ATP/UTP sensor, P2RY2 is sensitive to microenvironmental ATP levels, which are known to be elevated in vivo compared to cell culture conditions. This discrepancy could account for why an earlier selinexor-induced activation of AKT was observed in vivo versus in vitro (FIG. 2B, FIG. 2C).

### P2RY2 is Required for Selinexor-Induced Activation of AKT

To assess the role of P2RY2 in selinexor-induced AKT activation, the ability of P2RY2 to activate AKT in the AML cell lines was first tested by culturing cells in the presence of exogenous ATP or UTP. Cells cultured with exogenous ATP activated AKT signaling while cells cultured with exogenous UTP only minimally activated AKT, if at all, prompting the possibility that selinexor may activate AKT signaling through induced release of extracellular ATP. This was tested by treating cells with selinexor versus vehicle in the presence of the ectonucleotidase inhibitor ARL67156 and quantifying the amount of extracellular ATP released after 36 hours. After normalizing for slight differences in cell quantity, there was no increase in extracellular ATP in cells treated with selinexor, suggesting that AKT activation is predominantly due to upregulation of P2RY2, not an increase in the abundance of its ligand.

To verify the role of P2RY2 in selinexor-induced AKT activation, doxycycline-inducible shRNAs were used to knock down P2RY2 and subjected those cells to selinexor treatment. Using two different inducible shRNA constructs, P2RY2 knockdown blunted the capacity of selinexor treatment to activate AKT (FIG. 4C). This effect was phenocopied using AR-C118925XX (AR-C), a selective P2RY2 antagonist, and pertussis toxin (PTX), which uncouples G protein-mediated chemokine signaling through ADP-ribosylation of Gi and Go (FIG. 4D). These data indicated that selinexor's activation of AKT was P2RY2-dependent. Interestingly, it was observed that P2RY2 inhibition or knockdown precluded AKT activation in both FLT3 mutant and wild-type AML cell lines. Given the known crosstalk between GPCR and RTK signaling and the ability of oncogenic FLT3 to constitutively activate AKT, it was sought to determine whether FLT3 and selinexor-induced P2RY2 signaling were behaving as interacting or orthogonal inputs into AKT. To test this, an activating D835Y-mutant FLT3 or GFP was lentivirally expressed in OCI-AML2 cells and treated with AR-C. In the cells expressing GFP, western blot analysis revealed that AR-C inhibition of P2RY2 suppressed p-AKT T308, suggesting that a substantial component of baseline AKT signaling can be attributed to P2RY2. Exogenous expression of D835Y-mutant FLT3 promoted p-AKT T308; treatment with AR-C reduced but did not completely ablate the elevated p-AKT T308 signal, suggesting that AKT can receive simultaneous activating inputs from both P2RY2 and FLT3.

Because PIK3CG and PIK3R5, which encode subunits of the Gβγ-receptive PI3Kγ, were the only two PI3-kinase subunits that scored in both the FACS and sensitizer screens, it was reasoned that PI3Kγ may be responsible for enabling P2RY2-driven activation of AKT. Using inducible shRNA constructs, it was shown that, like knockdown of P2RY2, knockdown of PIK3CG or PIK3R5 (but not PIK3CA, PIK3CB, or PIK3CD) blunted activation of AKT (FIG. 4E). This effect was phenocopied using the PI3Kγ-specific inhibitor IPI-549. Together, these data show that PI3Kγ plays a vital role in selinexor-induced AKT activation.

### Selinexor-Induced Activation of AKT Requires Activation of RAS

In order for PI3Kγ to be fully responsive to Gβγ, not only must p110γ associate with p101, it must also associate with activated RAS (GTP-RAS). To that end, it was postulated as to whether selinexor-induced P2RY2 signaling activated RAS as an accessory to full PI3Kγ activation. Several lines of experimental evidence were found in support of this model. First, in five AML cell lines, RAF1-ras-binding-domain (RAF1-RBD) pulldowns revealed increased GTP-RAS in selinexor-versus vehicle-treated samples, evidence that selinexor-treatment activated RAS (FIG. 4F). To determine whether P2RY2 was required for selinexor-induced RAS activation, cells were treated with selinexor in the presence or absence of AR-C. The addition of AR-C was able to reverse the increased GTP-RAS observed upon RAF1-RBD pulldown following selinexor treatment (FIG. 4G). Likewise, doxycycline-inducible shRNAs targeting P2RY2 were able to phenocopy the effect of P2RY2 inhibition on selinexor-mediated GTP-RAS loading (FIG. 4C). These data suggest that selinexor treatment activates RAS in a P2RY2-dependent manner.

To test the requirement of RAS for full selinexor-induced AKT activation, doxycycline-inducible shRNAs were used to knockdown KRAS and NRAS. Cells with simultaneous knockdown of KRAS and NRAS retained their viability but were unable to mount an AKT-activating response to selinexor treatment, suggesting that Ras is required for selinexor-induced activation of AKT (FIG. 4H). Interestingly, singular knockdown of KRAS did not blunt activation of AKT and knockdown of NRAS was insufficient to fully suppress selinexor-induced AKT activation. This observation may explain why none of the RAS isoforms scored in either CRISPR screen, although one cannot rule out noise, technical error, or compensation from nontargeted isoform(s), especially since CRISPR-based methods have been described to provoke isoform-specific, compensatory upregulation. Next, if selinexor treatment activates RAS, it could be expected to activate the mitogen activated protein kinase cascade. Indeed, RPPA analysis of selinexor-versus vehicle-treated cells confirmed an increase in phopho-ERK at Thr202/Tyr204. Western blot analysis of multiple AML cell lines corroborated that selinexor activated MAPK signaling in a P2RY2 dependent manner. To determine whether these trends would hold up in larger datasets, data was analyzed from a published gene expression dataset representing 526 patients diagnosed with de novo AML using ssGSEA and observed concordance between enrichment for the selinexor treatment gene signature and multiple predefined RAS/MAPK pathway signatures. Finally, it is worth mentioning that, in contrast to non-small-cell lung cancer, where XPO1 was previously reported as a KRAS-specific dependency, the findings suggest that in AML cells, selinexor-provoked activation of RAS promotes resistance to selinexor through activation of AKT. This is supported by experimental data showing that exogenous expression of oncogenic RAS and AKT mutants confer resistance to selinexor and publicly-available genetic dependency data, which reveal no relationship between the RAS mutation status of AML cell lines and their response to selinexor. Together, these results are consistent with a model where selinexor treatment promotes the upregulation of P2RY2, coordinately activating both PI3Kγ and RAS, which in turn empowers PI3Kγ to fully activate AKT.

### AKT Inhibition Sensitizes AML Cell Line Models to Selinexor Treatment

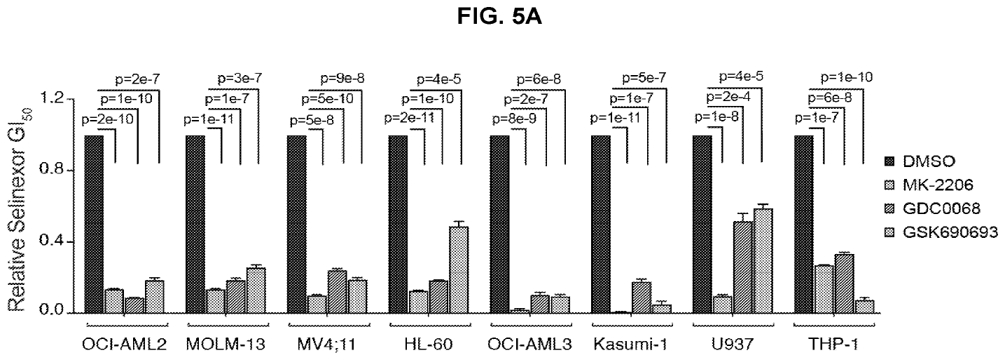

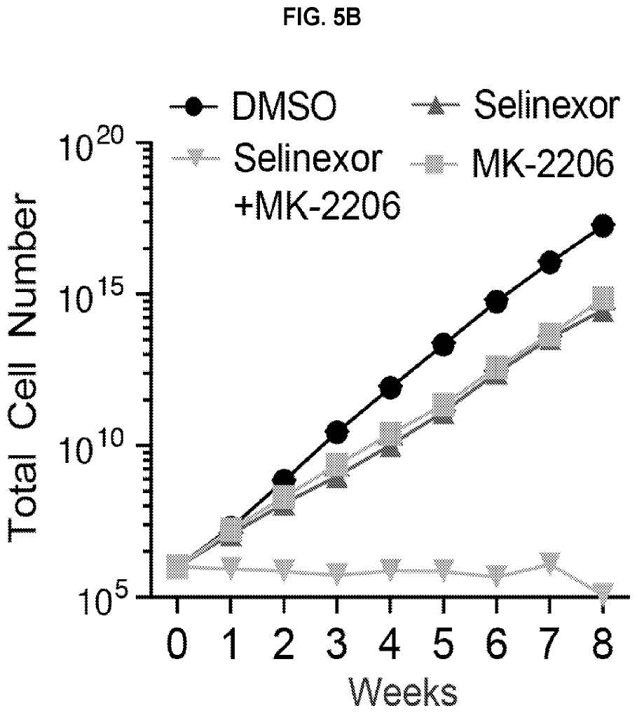

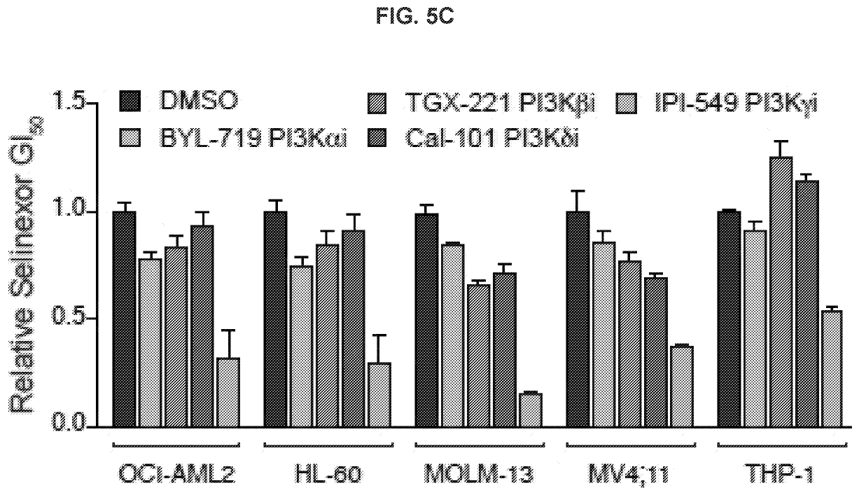

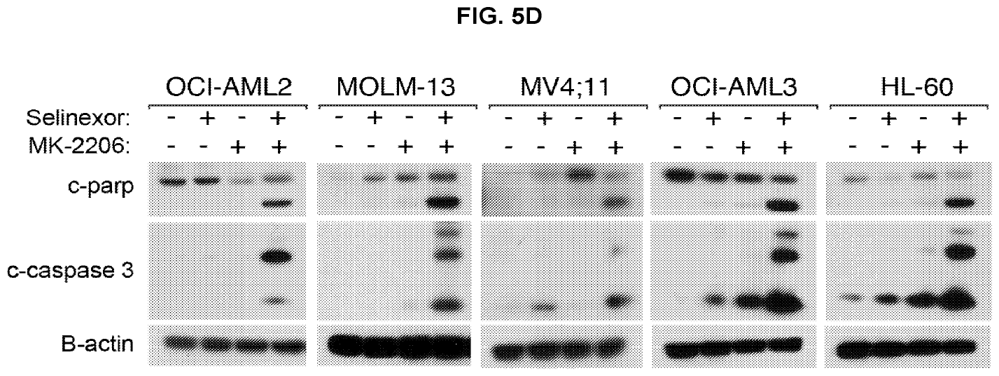

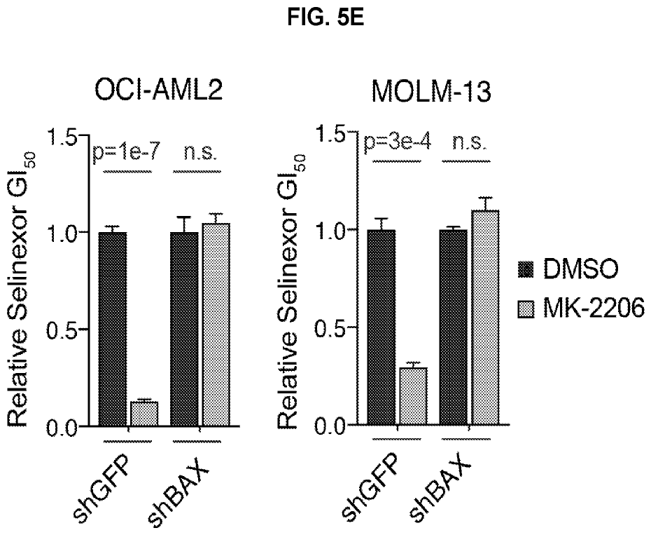

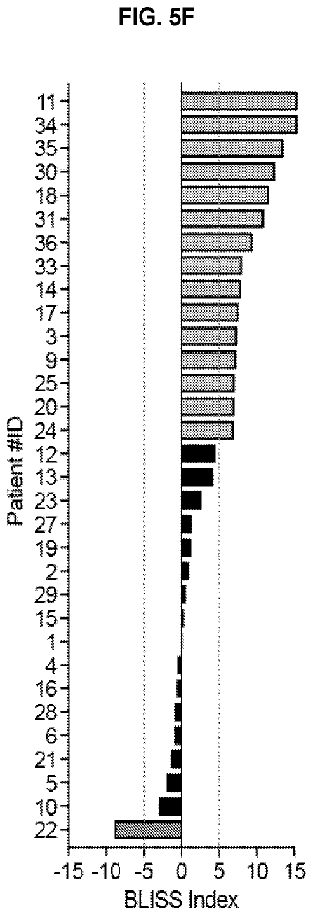

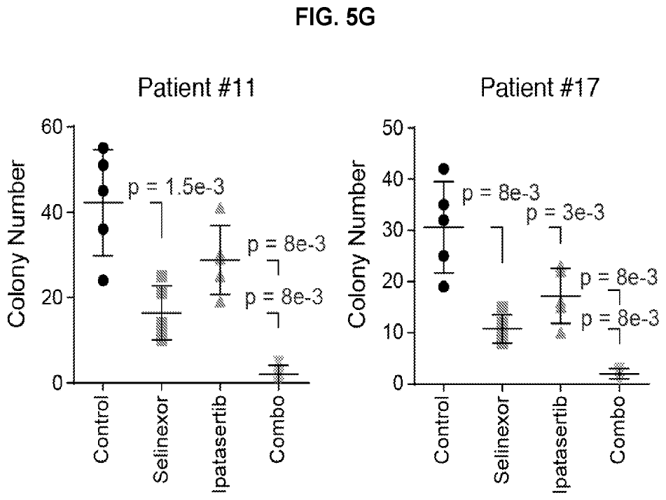

Having determined that selinexor treatment activates AKT signaling in a P2RY2 and p110γ-dependent manner, it was sought to explore AKT as a drug target whose inhibition is capable of potentiating selinexor's anti-leukemic effects. To do this, a panel of eight AML cell lines were treated with selinexor in combination with three AKT inhibitors: MK2206, GDC-0068, and GSK690693. Because PIK3CG expression varies in AML, cell lines were selected that represent a breadth of PIK3CG levels. Each of the AKT inhibitors were capable of sensitizing all eight of the AML cell lines tested to selinexor (FIG. 5A). The ability of both allosteric and ATP-competitive AKT inhibitors to sensitize cells to selinexor implies that the sensitization effect is on target to AKT. MK-2206 was selected for in vitro follow up studies. Bliss criteria confirmed synergy between MK-2206 and selinexor across a wide range of drug doses. This is consistent with the ability of shRNA-mediated XPO1 knockdown to sensitize cells to treatment with MK-2206. The same combination was shown to be capable of forestalling the outgrowth of selinexor resistance over 8 weeks of continued drug exposure (FIG. 5B). Last, because selinexor treatment also activates MAPK signaling, it was tested whether inhibition of MEK or ERK was capable of sensitizing AML cells to selinexor. It was found that MEK/ERK inhibition was capable of sensitizing AML cells to selinexor to varying degrees, but to a lesser extent than AKT inhibition. This is consistent with the degree of selinexor-induced pathway activation observed and with how MAP2K1 (MEK) and MAPK3/1 (ERK1/2) scored in the sensitizer screen.

Given the requirement of P2RY2 and PI3Kγ signaling for AKT activation, it was reasoned that targeting either P2RY2 or PI3Kγ should also be capable of sensitizing AML cells to selinexor. To test this, doxycycline-inducible shRNAs were used to knock down P2RY2 in OCI-AML2 and MOLM13 cells and treated them with selinexor. Knockdown of P2RY2 was capable of sensitizing both cell lines to selinexor. Next, AML cell lines were treated with selinexor in combination with the PI3Kα-specific inhibitor BYL-719, the PI3 KB-specific inhibitor TGX-221, the PI3Kδ-specific inhibitor Cal-101, and IPI-549. IPI-549 was capable of sensitizing each AML cell line to selinexor while the α/β/δ-specific inhibitors were unable to sensitize any of the cell lines to selinexor (FIG. 5C).

Next, the ability of AKT and CRM1 co-inhibition to provoke apoptosis was assessed. Similar to the potentiation of selinexor-induced PARP cleavage afforded by shRNA knockdown of P2RY2 (FIG. 4C), western blot analysis revealed that AKT inhibition promoted selinexor-induced cleavage of PARP and caspase 3 across cell lines (FIG. 5D). These findings were corroborated by annexin staining and suggested that the observed drug synergy was due to greatly increased potentiation of drug-induced apoptosis. To confirm this, shRNA constructs were used to knockdown BAX before treating cells with the AKT inhibitor and selinexor combination. BAX knockdown was capable of fully rescuing the synergistic effect of AKT inhibition on selinexor activity (FIG. 5E).

As a model for clinical disease, the combination of AKT inhibition and selinexor was tested in primary patient-derived AML samples. For these and subsequent experiments, the AKT inhibitor GDC-0068 (ipatasertib) was used due to its relative clinical maturity. To assess for pharmacologic synergy between selinexor and ipatasertib, each of a panel of 32 AML patient-derived samples was treated with a drug-dilution matrix comprised of 88 different selinexor and ipatasertib dose combinations for 24 hours before quantifying the cell viability corresponding to each dose combination. Next, each viability matrix for Bliss synergy was analyzed. Mathematical Bliss synergy was observed in 24 of the 32 samples; significant synergy across the dose-synergy landscape, identified by an average Bliss score greater than five, was observed in 15 of the 32 samples (FIG. 5F). Using a focused, 38-gene, next generation sequencing panel, the samples were genotyped and a trend was identified suggesting that cells with mutations in cohesin factors may exhibit heightened synergy when treated with the combination. This observation is preliminary but could serve as a basis for further investigation, underscoring the importance of subsequent, larger clinical studies enrolling AML patients with diverse genetic backgrounds.

Last, to additionally verify the effectiveness of combining selinexor with ipatasertib in primary cells, samples were taken from two of the patients for which adequate additional cells were obtained, independently cultured them in methylcellulose, and treated the cells with selinexor or ipatasertib or the combination. In both cases, significantly fewer colonies formed under conditions treated with the combination were observed than those treated with either agent alone, consistent with the synergy previously observed in media (FIG. 5G).

### AKT Inhibition Sensitizes Multiple Murine Models of AML to Selinexor Treatment

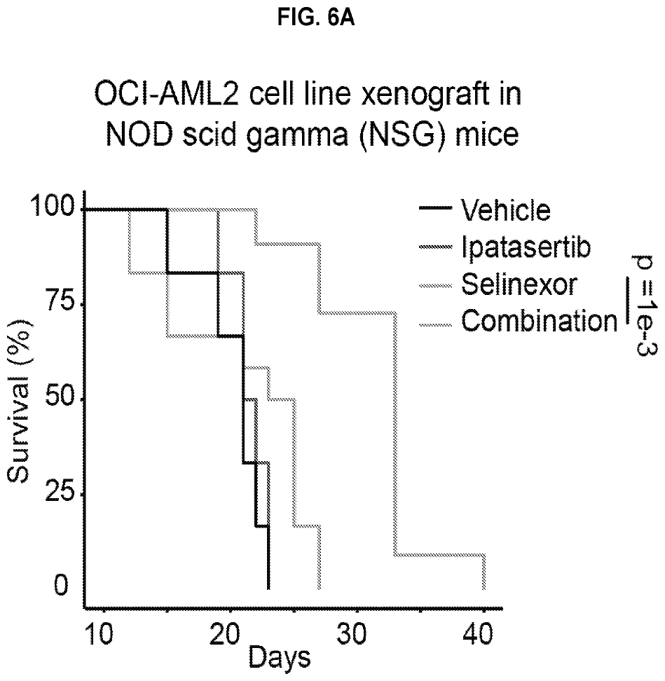

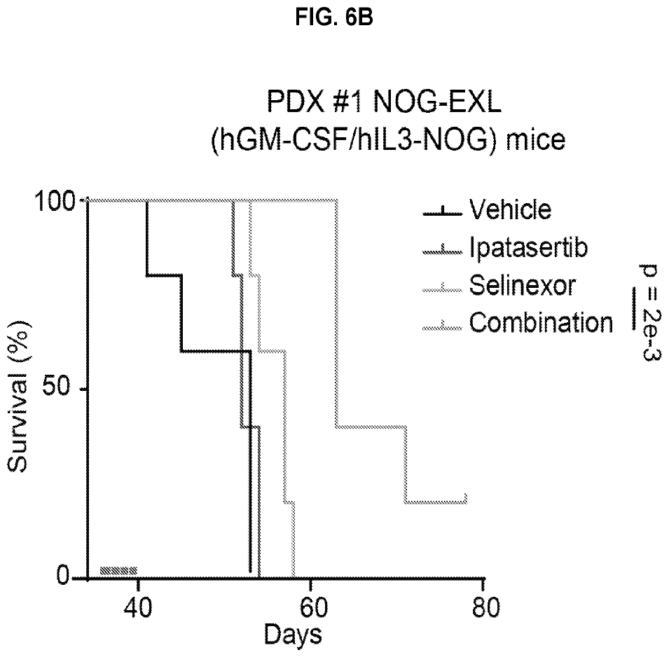

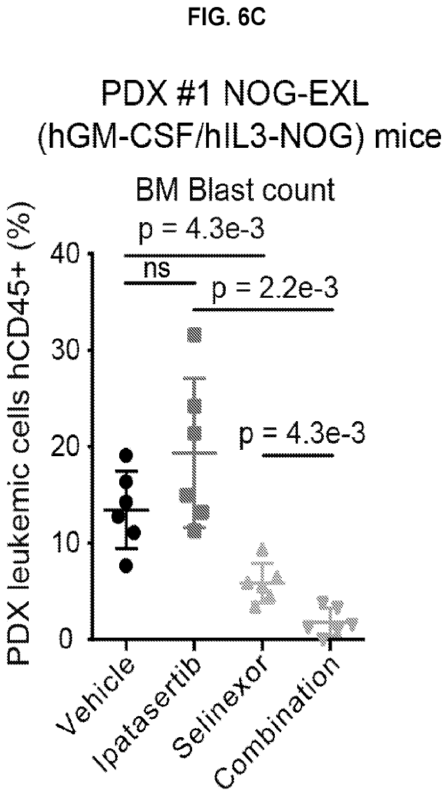

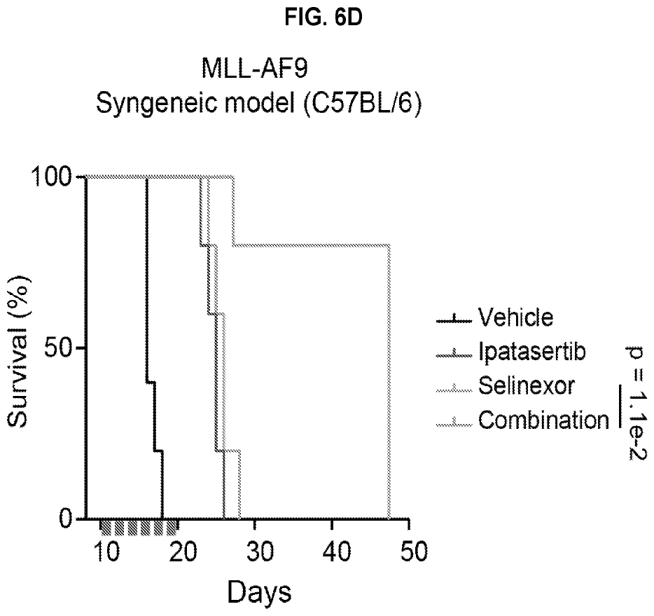

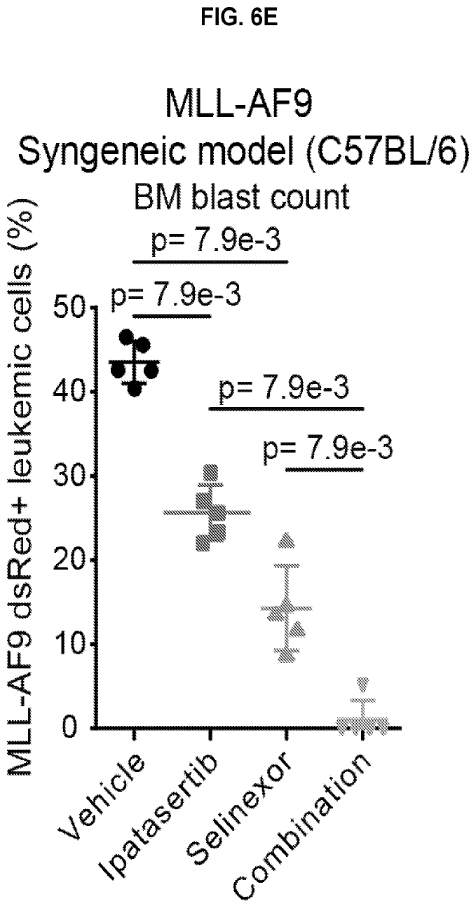

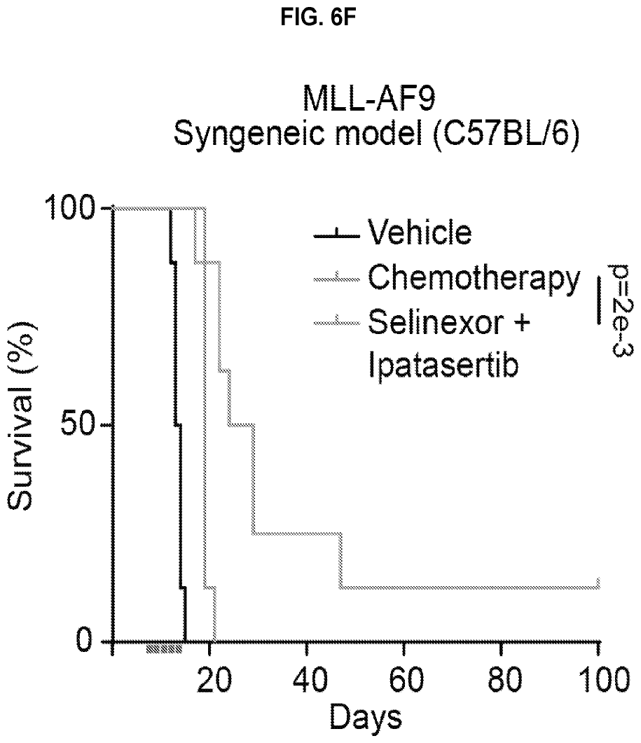

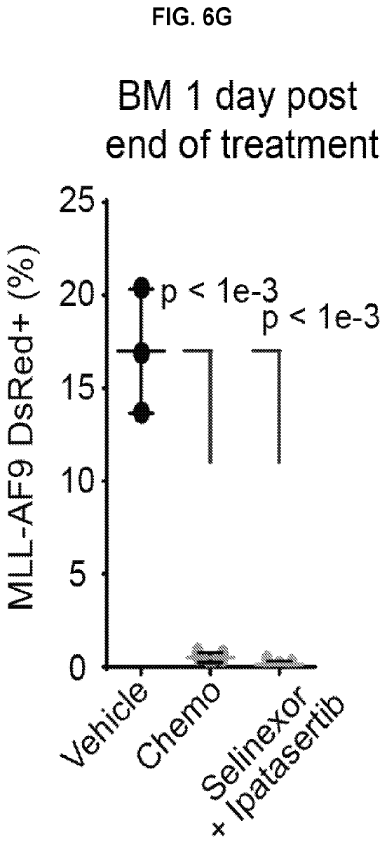

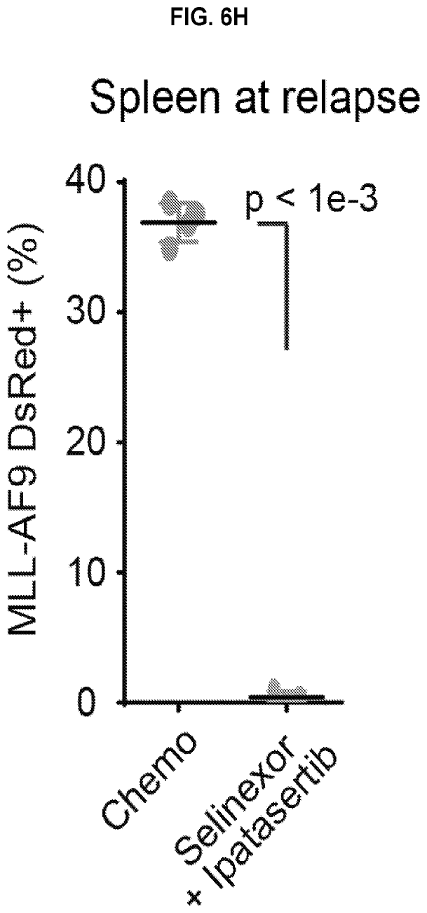

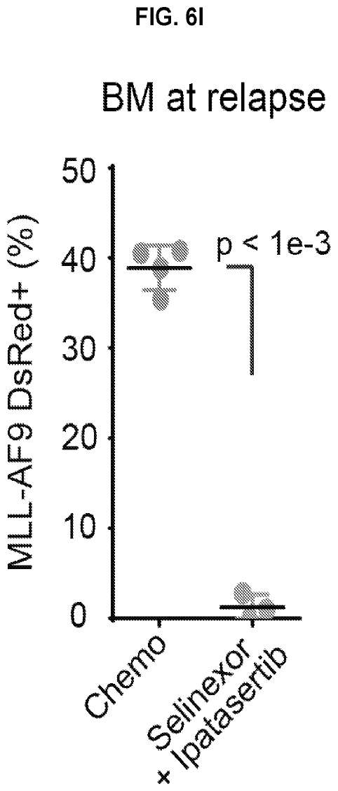

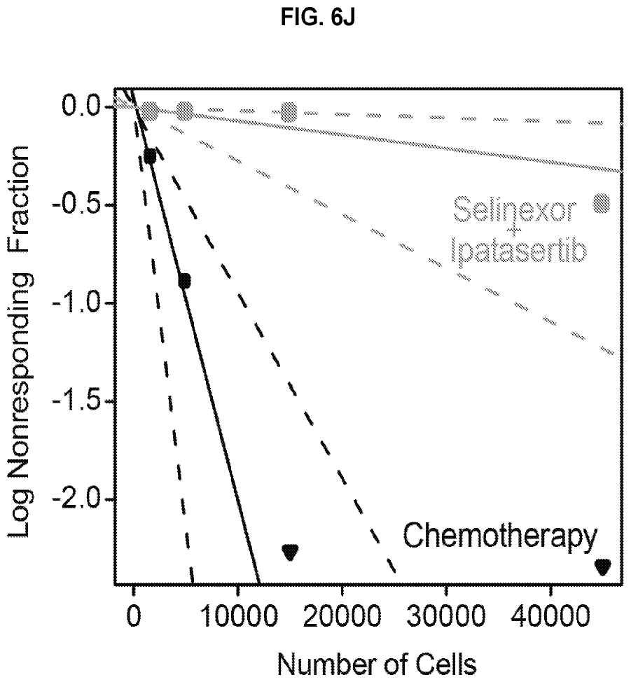

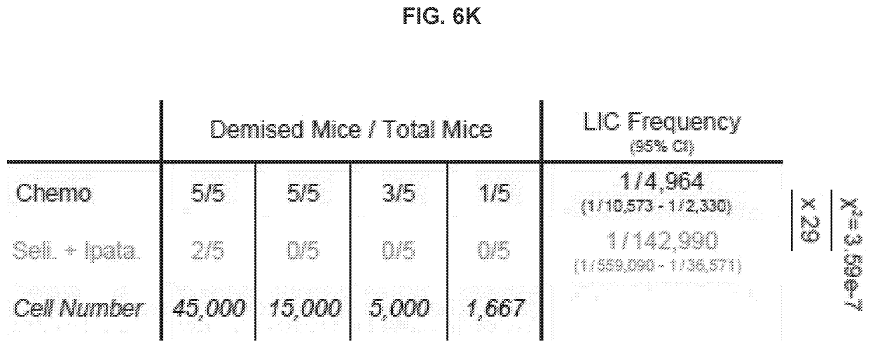

To assess the effectiveness of combined AKT inhibition and selinexor treatment in vivo, the combination in three different murine models of AML were tested. First, an orthotopic cell line xenograft model of AML were evaluated, established by introducing OCI-AML2 cells into nonobese diabetic (NOD)-severe combined immunodeficiency (SCID) IL2R-γnull mice (NSG mice) by tail vein injection. Following successful engraftment, as determined by bioluminescence, mice were sorted into treatment groups and treated with control, selinexor (10 mg/kg, oral gavage), ipatasertib (75 mg/kg, oral gavage), or both selinexor and ipatasertib every other day for five days. In this experiment, the combination of selinexor and ipatasertib was capable of significantly prolonging survival beyond what was achievable with either drug alone (FIG. 6A).

Next, the same combination was tested using two patient-derived xenograft (PDX) models. First, the primary patient AML cells were tested in vitro across a range of selinexor and ipatasertib doses to establish the presence of pharmacologic synergy. The primary cells were then transplanted into NOD/shi-SCID/IL2R-γnull (NOG)-Tg(SV40/HTLV-IL3, CSF2)(EXL) mice (NOG-EXL mice), which constitutively expressed human GM-CSF and human IL-3 to support myeloid engraftment and reconstitution. PDX engraftment was determined by the presence of blasts in the peripheral blood or bone marrow. Following engraftment, mice were randomized and treated with control, selinexor (15 mg/kg), ipatasertib (65 mg/kg), or both selinexor and ipatasertib every other day for one week. In both PDX models of AML, the combination of selinexor significantly prolonged survival beyond that of mice treated individually with either selinexor or ipatasertib (FIG. 6B). Flow cytometry analysis showed that mice treated with the combination of selinexor and ipatasertib exhibited the lowest percentage of human CD45+ leukemic blasts in comparison with animals treated with each drug alone (FIG. 6C). These data suggest that the addition of ipatasertib synergistically enhanced the anti-leukemic effect of selinexor.

To further validate the antileukemic potency of the selinexor and ipatasertib combination, an aggressive MLL-AF9-driven syngeneic model mouse model of AML was selected whose median latency for disease progression is only 14 days from the time of injection. Animals transplanted with these AML cells were treated with the combination of selinexor (15 mg/kg, oral gavage) and ipatasertib (65 mg/kg, oral gavage) or either drug alone every other day for ten days. Parenthetically, the selected 15 mg/kg dose of selinexor was the maximum tolerated dose in mice concomitantly receiving background doses of ipatasertib and was sufficient to elicit an on-target response, as evidenced by induction of p53. In the combination study, animals that received selinexor or ipatasertib survived, on average, no more than 10 days longer than those that received vehicle. In contrast, animals that received both selinexor and ipatasertib were able to survive nearly 30 days longer than those that received vehicle (FIG. 6D). The increased overall survival of mice treated with the combination of both agents was associated with a pronounced reduction of the MLL-AF9 blast proportion in bone marrow as compared with animals treated with each drug alone (FIG. 6E).

### The Combination of Selinexor and AKT Inhibition Outperforms Standard-of-Care Chemotherapy

In order to compare the effectiveness of selinexor and ipatasertib against a standard-of-care chemotherapeutic regimen based on the combination of cytarabine and anthracycline used to treat AML patients, a head-to-head experiment was conducted using the syngeneic MLL-AF9 mouse model treated with either vehicle, cytarabine (100 mg/kg) and doxorubicin (1 mg/kg), or selinexor (15 mg/kg, oral gavage) and ipatasertib (65 mg/kg, oral gavage) every other day for five days. In this aggressive murine model of AML, the combination of selinexor and ipatasertib conferred a significant survival advantage over maximally-tolerated doses of chemotherapy, which were minimally effective (FIG. 6F). Notably, one out of eight mice exhibited a sustained disease remission through 100-day follow up. Analysis of bone marrow samples taken from all cohorts one day after the end of treatment showed a profound decrease in the leukemic cells taken from the bone marrows of mice that received either combination, suggesting that both combinations were initially able to achieve bone marrow penetrance (FIG. 6G). Subsequently, analysis of bone marrow and splenic samples taken from each mouse at disease relapse (defined as the point of first relapse in either treatment group) revealed that the combination of selinexor and ipatasertib was capable of providing durable remissions while the combination of cytarabine and doxorubicin was not (FIG. 6H, FIG. 6I). Of note, in this experiment, administration of selinexor and ipatasertib was restricted to the five-day treatment schedule for cytarabine and doxorubicin. However, selinexor and ipatasertib would likely be more tolerable in patients than standard-of-care chemotherapy, and would, in theory, be dosed longitudinally, suggesting that the effects observed in this model may be underestimating their true clinical potential.

Last, multiple lines of evidence have suggested that resistance to front-line therapies in AML is a consequence of the re-expansion of leukemia-initiating cells (LICs)—empirically defined by their ability to seed leukemia in animal hosts—which subsequently fuel disease relapse. Therefore, therapeutic strategies that are clinically efficacious and durable theoretically require the use of anticancer drugs that are capable of eradicating the LIC fraction. To assess the ability of selinexor and ipatasertib to target LICs, MLL-AF9 leukemic cells were harvested from mice treated with either vehicle, the combination of selinexor and ipatasertib, or the combination of cytarabine and doxorubicin, each dosed as above. 24 hours after initial treatment, the leukemic burdens in mice treated with the three combinations was assessed by flow cytometry. Leukemic cells were sorted for by DsRed, serially diluted to establish accurate cell concentrations, and reinjected into sublethally-irradiated recipient mice. Using extreme limiting dilution analysis, a 29-fold decrease in LIC frequency in secondary recipients injected with blasts harvested from selinexor- and ipatasertib-treated donor mice was observed compared to those engrafted with blasts harvested from cytarabine- and doxorubicin-treated donor animals (FIG. 6J). As a result, the survival of mice injected with blasts pretreated with the combination of selinexor and ipatasertib was substantially prolonged, at any cell concentration tested, compared to the chemotherapy-pretreated group (FIG. 6K).

It is reported herein that treatment of AML cells with the nuclear export inhibitor selinexor activates the pro-growth and pro-survival PI3K/AKT pathway through transcriptional upregulation of the purinergic GPCR, P2RY2. Using in vitro, in vivo, and patient-derived model systems, it was demonstrated that co-inhibition of AKT potentiates the anti-leukemic effects of selinexor.

Principally, the data nominate the combination of selinexor and AKT inhibition for the treatment of AML. This drug combination was widely synergistic, as evidenced across multiple cell lines, AKT inhibitors, and assays. Most notably, in three murine models of AML—a patient xenograft model, a cell line xenograft model, and a syngeneic mouse model—treatment with selinexor and an AKT inhibitor significantly prolonged survival versus selinexor alone. Further, when cotreatment with selinexor and ipatasertib was tested against an established chemotherapy regimen in a syngeneic MLL-AF9-driven mouse model of AML, ipatasertib and selinexor outperformed the standard-of-care combination by a wide margin. These results are particularly striking in light of selinexor's clinical history. Initial efforts to treat AML patients with selinexor as monotherapy produced only modest results, perhaps partially restricted by the activation of oncogenic signaling that is describe here.

More broadly, this work begins to reconcile contrasting notions of how anticancer therapies affect cell fitness: one which holds that therapies monotonically restrict cancer cell fitness, and another, which reserves the possibility that anticancer drugs may elicit pro-fitness effects that are separable from their anti-fitness effects. The study reveals that selinexor treatment compels the on-target activation of downstream signaling cascades that simultaneously restrict and support AML cell fitness. Importantly, these effects are not compensatory, and are therefore set apart from the well-described ability of cells to adaptively respond to drug-induced stress; they represent on-target sequelae of CRM1 inhibition, and are therefore distinct from off-target effects that can limit the application of small molecules; and they exhibit determinism at the level of each cell, and thus do not represent rare, subclonal events that are gradually selected for over time in the presence of drug.

What is claimed:

1. A method for treating a cancer in a subject in need thereof, the method comprising administering to the subject an effective amount of a first agent that inhibits cellular nucleus export and an effective amount of a second agent that inhibits Protein kinase B (Akt).

2. The method of claim 1, wherein the first agent inhibits exportin 1.

3. The method of claim 1, wherein the first agent comprises Selinexor, Eltanexor, or a combination thereof.

4. The method of claim 1, wherein the second agent inhibits Akt via inhibiting a G-protein coupled receptor (GPCR), a phosphoinositide 3-kinase (PI3K), or a combination thereof.

5. The method of claim 4, wherein the second agent inhibits Akt via inhibiting purinergic receptor P2RY2, phosphoinositide 3-kinase gamma (PI3K γ), or a combination thereof.

6. The method of claim 1, wherein the second agent comprises MK-2206 2HCl, Perifosine, GSK690693, AZD5363, Ipatasertib, Capivasertib, PF-04691502, AT7867, Tricirbine, CCT128930, A-674563, PHT-427, Miransertib, BAY1125976, Borussertib, Miransertib, Akti-1/2, Uprosertib, Afuresertib, AT13148, Oridonin, Miltefosine, Honokiol, TIC10 Analogue, Urolithin B, Resibufogenin, Cinobufagin, Daphnoretin, Loureirin A, Trigoneline, ML-9 HCl, ABTL-0812, Alobresib, Praeruptorin A, Oroxin B, SC66, Usnic acid, Scutellarin, Astragaloside IV, Deguelin, TIC10, Methyl-Hesperidin, IPI-549, TAS-117, ARQ-751, LY2780301, or a combination thereof.

7. The method of claim 1, wherein the second agent comprises Ipatasertib.

8. The method of claim 1, wherein the cancer comprises leukemia, lymphoma, myeloproliferative neoplasms, myelodysplastic syndromes, amyloidosis, Waldenstrom's macroglobulinemia, aplastic anemia, myeloma, or solid cancers.

9. The method of claim 1, wherein the cancer comprises acute myeloid leukemia (AML).

10. The method of claim 1, wherein the first agent is administered concurrently with the second agent to the subject.

11. The method of claim 1, wherein the second agent is administered to the subject after the first agent is administered.

12. The method of claim 1, wherein the second agent is administered at least 8 hours after the first agent is administered.

13. The method of claim 1, wherein the effective amount of the first agent comprises about 0.1 mg/kg to about 100 mg/kg, the effective amount of the second agent comprises about 0.1 mg/kg to about 100 mg/kg, or a combination thereof.

14. The method of claim 1, wherein the first agent and the second agent are administered as a single dosage form.

15. The method of claim 1, wherein the subject is a mammal.

16. The method of claim 1, wherein the second agent inhibits a pro-oncogenic effect of the first agent.

17. The method of claim 1, wherein the method decreases a number of CD45+ cancer cells in the subject compared to an administration of the first agent alone or the second agent alone.

18. The method of claim 1, where the method increases a time of survival of the subject compared to an administration of the first agent alone, the second agent alone, or cytarabine and doxorubicin.

19. The method of claim 1, wherein the method decreases a number of leukemia-initiating cells in the subject compared to an administration of cytarabine and doxorubicin.

20. A composition comprising:
a first agent that inhibits cellular nucleus export; and
a second agent that inhibits Akt; and
one or more pharmaceutically acceptable excipients.

21. The composition of claim 20, wherein the pharmaceutically acceptable excipients comprise buffering agents, solubilizers, solvents, antimicrobial preservatives, antioxidants, suspension agents, a tablet or capsule diluent, or a tablet disintegrant.

22. The composition of claim 20, wherein the first agent inhibits exportin 1.

23. The composition of claim 20, wherein the second agent inhibits Akt via inhibiting purinergic receptor P2RY2, phosphoinositide 3-kinase gamma (PI3K γ), or a combination thereof.

24. The composition of claim 20, wherein the composition comprises about 1 mg to about 800 mg of the first agent, comprises about 1 mg to about 800 mg of the second agent, or a combination thereof.

25. The composition of claim 20, wherein the second agent inhibits a pro-oncogenic effect of the first agent.

26. A kit comprising:
a first agent that inhibits cellular nucleus export;
a second agent that inhibits Akt; and
one or more packages, receptacles, delivery devices, labels, or instructions for use.
